SEQUENCE LISTING (84 sequences)

SEQ ID NO: 1 - DNA, 40 bp, Artificial Sequence
  conserved sequence 1
tgattacgtccctgccctttgtacacaccgcccgtcgcta

SEQ ID NO: 2 - DNA, 68 bp, Artificial Sequence
  conserved sequence 2
aaacttggtcatttagaggaastaaaagtcgtaacaaggtttccgtaggtgaacctgcggaaggatca

SEQ ID NO: 3 - DNA, 148 bp, Artificial Sequence
  conserved sequence 3
actttcaacaacggatctcttggytyycrcatcgatgaagaacgcagcgaaatgcgataagtaatgtgaattgcagaattcmgtgaatcatcgaatctttgaacgcammttgcgcccyttggtattccgaagggcatgcctgtttgrg

SEQ ID NO: 4 - DNA, 47 bp, Artificial Sequence
  conserved sequence 4
acccgctgaacttaagcatatcaataagcggaggaaaagaaacyaac

SEQ ID NO: 5 - DNA, 32 bp, Artificial Sequence
  conserved sequence 5
cccgtcttgaaacacggaccaaggagtctaac

SEQ ID NO: 6 - DNA, 90 bp, Artificial Sequence
  16S conserved sequence
caccggctaactccgtgccagcagccgcggtaatacggagggtgcaagcgttaatcggaattactgggcgtaaagcgcacgcaggcggtt

SEQ ID NO: 7 - DNA, 50 bp, Artificial Sequence
  16S conserved sequence
gtggggagcaaacaggattagataccctggtagtccacgccgtaaacgat

SEQ ID NO: 8 - DNA, 80 bp, Artificial Sequence
  artificial sequence (a2)
attgtcagtctagcgaatcattataccgaagaacatccgtttatgagaacgtgctaccaattaactgtactaagctgtcc

SEQ ID NO: 9 - DNA, 80 bp, Artificial Sequence
  artificial sequence (a2)
ttactgatcgaacgtcgtataatgctgaggcatctgttattaaccgtacctttcaaggattaccatgtggcaacataagt

SEQ ID NO: 10 - DNA, 80 bp, Artificial Sequence
  artificial sequence (a2)
ttggccttcagtcgagaacttgttgaaactgtcctgacgcactggaacgagcttccattgattcgctagaaatgccgacc

SEQ ID NO: 11 - DNA, 80 bp, Artificial Sequence
  artificial sequence (a2)
cctagaaagctcgccattagccgcagtagtgattggacatcagagtttcgctcacaacgtcaccgctcgttatggaactt

SEQ ID NO: 12 - DNA, 80 bp, Artificial Sequence
  artificial sequence (a2)
tcaggaagtgtgtcccattgccggaggagtcctattgaatcacggattacgtctgtaacgctggaccgaggttgtatcat

SEQ ID NO: 13 - DNA, 80 bp, Artificial Sequence
  artificial sequence (a2)
tcccgcaaatacctttggagtgcgtcactatctaggagtgtgccgatgactcgtaatctccatcctcgaagttgcacgat

SEQ ID NO: 14 - DNA, 80 bp, Artificial Sequence
  artificial sequence (a2)
gacaccctgttcagattagcgagcctcagttacaccagattccgagttcgtaagatcgagaggagccatcatggacgttt

SEQ ID NO: 15 - DNA, 80 bp, Artificial Sequence
  artificial sequence (a2)
catgactggaaaccctctgacgtgtaactctggaagctcagttatcggaaacggcgctaagctacgtgatcgtaagcagt

SEQ ID NO: 16 - DNA, 80 bp, Artificial Sequence
  artificial sequence (a2)
gcacctagcctttaacgagaagaatgtagccctacgccatcggcatgtgattccatacgatgttacgaaacctgaggcag

SEQ ID NO: 17 - DNA, 80 bp, Artificial Sequence
  artificial sequence (a2)
tgcggagcatcctagtacaatatccggttgcctataagcccggtatgcgcgaattaacctaactgccagagatgagttcc

SEQ ID NO: 18 - DNA, 80 bp, Artificial Sequence
  artificial sequence (a2)
acggcactgatgttcacccgccgtcgatcatacacgcagggcgatgactctatgcgaggctccgaccagtaacaggcgct

SEQ ID NO: 19 - DNA, 80 bp, Artificial Sequence
  artificial sequence (a2)
cgtacctgtcagcacgctgttgaccttagcccgtggcaacgactgtgaagcctccgacacgtactgagggcgattcccag

SEQ ID NO: 20 - DNA, 360 bp, Artificial Sequence
  artificial sequence (b2)
tcataagcagagcctttatcccatataagctattgtcacgaagtgtcactgtgaacgaatgttctctaaacttactacggcttcagatgtaacggattcagactactctattcataacggactacagattgcgtcaactacgatattctcttgagatcacgattagcaagtacctttgcagcttgaaattaaccagacctttccttggaatgcctatacagagatttatcataccaggagttctccagattacctagatgtcttaacgagatacaggacttacacgatgacttagtgtgttgtttgcatcaacctaacagtaactgagcgaattgtaccaacgtattctttaccggaagt

SEQ ID NO: 21 - DNA, 120 bp, Artificial Sequence
  artificial sequence (b2)
catccttggtctaagaaagtgcatgatttgagcataccaatcgccattacgataaagatcctttgagtctaacgtacactgtgtcatctgtaagataccattgtcactacttcagtcaga

SEQ ID NO: 22 - DNA, 360 bp, Artificial Sequence
  artificial sequence (b2)
cacagtgtggatctgacgaattaccaaggcactccatgtgtgccatctacgtctcaggaattgtacctgctaccactaggcatcgagaacgctgcatgtattcaccgagtaaggtcttccagactccgataccgtatgtgttcccaggagaaatgtcgcttagccggttcaagccatcatgtgctagactagacacgtctatcgcggtttacacgaccatcagttgagccaatgctatccttgcgggtcaaacagagcttacggatcacccatagttgtcacgccacgttaaagttccgagcgaaacgctatctcttcgagagctgtcccaatgaaactctgcacggacttgtattgcac

SEQ ID NO: 23 - DNA, 360 bp, Artificial Sequence
  artificial sequence (b2)
aagcgttggttcgttacgcaaggctctacgaaagcagtgtctacttagcgttcagtgcagcgatccacaatctcatgggtatgtcatcgaccagctacgacgcaagtttcccagatcaagattaggtgcccttcaagcacggttggaactctaccgacaattacgaggtcccaattacgggtggcaactatgctgtaccagtaagatcctgccgattcgacgcacagtcataactcagtgtacgtgtatcctggcaaggaggaagctccctttacatgctagtgcaatgtccgcagtttgcgagaggactatatccagtctaccacaggtcagaggttacaccctggctatctagtatgg

SEQ ID NO: 24 - DNA, 360 bp, Artificial Sequence
  artificial sequence (b2)
gcttcgattacgatgcccaaatacgatccgcgtagtttccacgaggtctacagtaccctattgttcgaggcagtaacctgaaccgcgtctgtcaacagttatgtgacggcaagttgtccaagtccgagccatactatcagtcgtcttagctcatgggaagctcgcagtgttaagctcagtaggcaaattccagcgtgatgccgatccagtgtacgagaatccttacatgcaagtgtcgcaggccagatcagtttcgagaaagagtacgttctatccctggcgtcctcagtgactcaagatgagattacatccacacggtctcggtccattcgcaaagtacagtgtttccttagcagcagg

SEQ ID NO: 25 - DNA, 360 bp, Artificial Sequence
  artificial sequence (b2)
ataatccagggtccacgagtgaatgccctgcaaatgtaccaagttcctgaccttctggcatgtgaagccgatcttatcgctgaagagtctcgaagtcgctgacatacacccgtattgtcgatctgttggcgtaacggacatacgatgcactgacagcagttgcttagagcctagacacgacattgccttgaacgaccttgctactcatagggatacccgacgtagacgtttagtcctgcaagtcgaaagccctttgtgagagtcgccttatagtaccggatagtctcccagccatattggagagtccatatagccacggtagaatgctccgaggtaacctgagtcaaattgccgcactag

SEQ ID NO: 26 - DNA, 360 bp, Artificial Sequence
  artificial sequence (b2)
ctgacggaccaatctgtatgtaaagcggctattcaggagcctatccgacgagttgatgcttacaaggcgatctatccctgaccagtgctaaccatgtgcataagagcagtctcactcacgagtctcggttccttagacgattcaatgccaagttgtgccggagaacacctgttgatcctcgacaatgattcagtccaccgggatgtctgtagttcccaacgccaatatgtagagcttcggtccacgaaagtaccgtggtagccatgatatgacttacgcccgacaaagttcgggagtttctcgcatgtgaagtttccgcaaccatgagcaaggtcgtttgacctggaagtgtatgatccg

SEQ ID NO: 27 - DNA, 360 bp, Artificial Sequence
  artificial sequence (b2)
ctctgatggacctggtgatacacggtactatttggcatggtcacatcgggcatctgtaagacctccagttgtagtgtgcagagttcccagacagtctaagacggcattgactatggccttgtggttcgagaaccgaacatccaagagtttcgctcgttcatggcgataacccttcaacgtgtggtaacctgtaacgcagtcagctttagcgcgtgaataccttgaggcaatacaccgagttgtgctaccctagtgatgacagaatggcaccttatgctccggtacacctacggaatcatgcaagtggaatccctttcgagagcaggctcagtttagttgcgaagtgatctccgcatttcc

SEQ ID NO: 28 - DNA, 120 bp, Artificial Sequence
  artificial sequence (b2)
cttctgaaactatgacgcgccaaccggaatcgtgtaatggattgacctacttgctcggacgacggataacgctgtatgcaaatgtgcctgtaactcggctctgcgaactgctctgatcta

SEQ ID NO: 29 - DNA, 120 bp, Artificial Sequence
  artificial sequence (b2)
taggtcacgctagtaccaaggagactcagaccttacagcttgcttgcagacagatcggaatcccacagcagagtttagacgtttggagacagtcccacttcagtcgttggatgcacttag

SEQ ID NO: 30 - DNA, 360 bp, Artificial Sequence
  artificial sequence (b2)
cctggcgaatgtctaaggcgtccatatccgaggtgcagcgcgttgcctgaccattaggcccgtatagttcggcgtgaccgagatgccgctcagtacgacggtctaacaagctggccgcacttgccaacctgtcgcggactgtcttaacggtggcccgacttgctaccacacccgtgggattgtgctacgaagcgtcccgaaggtcctcagcccaagagtcctgtagtgagtacccggagcctcgaccctgatgtgatccgaccagattggagccggtgaccctcagacggagtcaaggtcctacctgtgaagccctgacggcgtggattcctgctagagccaaggagagtgtcccgctac

SEQ ID NO: 31 - DNA, 120 bp, Artificial Sequence
  artificial sequence (b2)
ccatactgcgaatgggagccgccggaggtaagtcctttccctgatgaccttgcgcgtagggccgggtaagagcttctccactgactgtcaaccgtgggcacgccgaggatgctactcatg

SEQ ID NO: 32 - DNA, 220 bp, Artificial Sequence
  artificial sequence (c2)
agttgtctgccagaaatcattgaacattccgacgaatatcgacatggttgcttatctaagaccttaaacggtacttggttagctgatcgcaatacttgaaagacttgatcctgtacttacctggacacgatgtaataatctcacacagttatgagaagctggttgcacctaaatagtcaattagcacgtagtaacgtagacttgccactgatgaaacata

SEQ ID NO: 33 - DNA, 220 bp, Artificial Sequence
  artificial sequence (c2)
cattgaacacttcgtaaggtacacctatggatcaacgattaagtctcgataccgtaagatggtaactctagtcagtgataatcaacagcgtagtacattcgtaagcagtcttggacattactttctgagtgcaacattcaacgtctaaacgggttaaatctctcataacggaacttgtgtgcaacagatgctatatggtatgcaaatgcgatacactttg

SEQ ID NO: 34 - DNA, 220 bp, Artificial Sequence
  artificial sequence (c2)
actatgaggcccacagttacgaacgactagaccactgtcttacgagtgtcgcaccataagatggcgagtaatccgctcaatccactggttcctgagaaagagccggaaatctgaggtcattctgcccatgatagctggaaacacccgagtctctaagtgtgagtagcctgatctactgcaaacgcccgatacatatcgtgagagtctgctaggactgatc

SEQ ID NO: 35 - DNA, 220 bp, Artificial Sequence
  artificial sequence (c2)
accgtaaagctaggtcaggtcttcactgggcaacgacataatgggtaactcacttccagcctacatcagcggtgtcaaaggtagatgcctatcgtaccacccacaatgctctagggtttcagagaagctgtgtcttccgatggtcaccagatggattcgactcaaggtcatacaggagtgtcgcgtaacatagcctatgcaaccgttcggttaaggacgt

SEQ ID NO: 36 - DNA, 220 bp, Artificial Sequence
  artificial sequence (c2)
aacatgctgcgtagtacgtcgatcaccaagctatgagcgttgtcaaaggagtgtcaaccgacgagtccaggtttcatcaccttgctaggtatccacaggtgcattaggcggctaagtcttccacatcgtattgccgaagtgtatcgcccagacattcaagctgtcagaactctgcgttacagaacgtgccgtcaagattcaggctatcatccgtgaacca

SEQ ID NO: 37 - DNA, 220 bp, Artificial Sequence
  artificial sequence (c2)
agtgacagttcacggtagcagctaaatcttcgggcatcacgagtacatgagtctcccatcgttaatccagcaagccgatgtggagctatttcaacgggacgtatatgtcgtccatccgagttgcggactatctacagggtgaattatgcgactgactgccttgccactacgaaacagtgcgttcaaattgcgctaagggcgtgcgaatacttatgcaggc

SEQ ID NO: 38 - DNA, 220 bp, Artificial Sequence
  artificial sequence (c2)
atctgacagccttctacgagcctgctgaatcagatgaaccacttggtcgcaatgatcgcaaggtcgggtatatcttcacggttagatccgaactgctccactgggtacaacacactgacttggtaactcggtcatacacgtcgggaacataactgcctgtgatagcacgcactcttaggacagtcgcattctctaggtcatggaatagcgcaacatcgct

SEQ ID NO: 39 - DNA, 220 bp, Artificial Sequence
  artificial sequence (c2)
aacttagggagtatgccgtcgaacatcgctcgtgagtaacttatcgtgcggatacacctcgtacatgccactcggtacttagaatagctggtaacctccgatgctcgcaatgcgtagttctggattccaatggaccaacggtcattcctgggtgacaaagcaatctcctgtagcaggtcacagttctcgtctcgcagtaacgaagtcctcttacgtcatg

SEQ ID NO: 40 - DNA, 220 bp, Artificial Sequence
  artificial sequence (c2)
tccacgtaaatcagcgcgttatgggtctgacgtaagcacaagggtcctatacacgctactctggttatccctgagaagtcggttaccatgtcacacagtcaggctatatgccctcacgttgattcgagcgaagttactgcaccaagtctggcgtagttagtgttccgtagagcaagtcactcaatcccgagcaaagtgtcgtgatgctgttcagcaagac

SEQ ID NO: 41 - DNA, 220 bp, Artificial Sequence
  artificial sequence (c2)
cagggttccctagtaagtacgattccaatacgcgatccgaatgcggcgtttcctaagcaaggtataatctcctgacgaggagtcgggtccataaggtttccatagttcaccgtgagactgcgatggtctgccaatgttcacttcaagtccgtaagacacggcaagagcctagcatctgttcgttcagagtcatggtatcggacaactgcctgatcttcga

SEQ ID NO: 42 - DNA, 220 bp, Artificial Sequence
  artificial sequence (c2)
gcggacgatgcctttgtcgataatgctcccgctgtaggccagcgccaatcggctgtgcatttagcgaggtctcacgccagtgcgagtacgagccttcctcctaagcgttcggtcggacaggacatctggatcgcggaaccctaatcccgtgggacaccgtcacttggtcgatgcgcgtagcttgtcaccgcagggactgagaggtcaacccatgcgactg

SEQ ID NO: 43 - DNA, 220 bp, Artificial Sequence
  artificial sequence (c2)
ggcagctttacggttcccagtgcctaatgaggacgcctgggcggaatcgagccttcggaaagacatctgcagcacggtgcctgcaacctgtcggtgacgtatcaggacctggtgtccacccgttgtcagggcttccaaggtcaagcaagtggtgaccggccatgcgtggtcgcttcacagaacatcacggcagtcgccgtatcggcccgagtgagactag

SEQ ID NO: 44 - DNA, 560 bp, Artificial Sequence
  artificial sequence (d2)
gaacgattgaagatgtactcagatattcattgatgggcctacgtctacttactatgggaatgtaaatactctgttccagcctaaggttagctttgcgaatacaaatgttcttatcgacgcacagtcatacggattacgatcaagttaatggttactccctaccgattattgcatccagatcatattgagaggaatcacctgtacggtttagaaatcagctctactagaagacactattgccatacgtcaaattgcagtgagtttcaccaaatcatggagatgttacccagttagcatacaactctttgcacaagtgcataatgtagtccctatgtcacaaggttatacgaagcatgtcaaatcatcgcctttagttacgatgtagttccacaagcgaaattagtttccgaaatggtcaagcatccaagtttagctcgaatctttaaggagatactcgaagtgcctatattacggaggtattatcatgtagcaagcgttacctagcttattagtccacgaatcatgtgttagaagtcgtcaagttcatgttatcctaccag

SEQ ID NO: 45 - DNA, 560 bp, Artificial Sequence
  artificial sequence (d2)
gtaaagctattaaccggagtgaatccttcattaaagtcgcacaagctgtattaccgttacgcaacgtatttgattgaccatgtgaacagaagtaccctattgacctagattatgcagcaatgcctaagactatttgcctaattcgggctatttagaccaatcctccatgatgtatatcagtcaaggctagtttggaacatacacgaaagtccttatgtagtagagtgcaattctcgtatccttcaacagtgttatcgagtatcgaacgattatcctatgggtatccacttatagaacgtgtgtagactaacctgtaaacgatgtctctgaaagcaagactacttatctgagatcggatgtttaagacgctatgacaccattaacttatgccagtgctagtcattatgaccacgatttggaatttatggctatcgccactatgaaatgctaagctacctgaacaatttgtacgcagtgacagtagatcctttgatccagaacttattaagagctgaccctatgaaacgtgatgtcctattcattattacgggaaaccgtag

SEQ ID NO: 46 - DNA, 560 bp, Artificial Sequence
  artificial sequence (d2)
tcaggctatattgaggcaccgcctggctagtagattacgacagctataacttcgggcaagccggttgatccaactatcgaaacctcgttagagcagtgtgtggcctaatggcatactggaacctatctgttacgccgagaactcgtgagcaactcagtctcataaagtcatggtccgcactgatgctgcacaaagctaccgattgatacgttcgccgactgtgatgcgtgaatcattccgtcaaagtgtccacccgtgtaggcattggtatatcgaccgatccaagaagcgacgcttagtacgcgattacattgggcagatggtacagctcccataaacgctaggaactgttcgcaagagtcctgtgtcagagtcaaggataccgttcagaggcaaactgaccgtcattcgtgctaaacgatgtgatccgccctttcagacgctagtgttacctggaagaagattggcgctacctatgtcccatacagcgacaaggtcttgtagaaggcatgtcaagctccctaaatggctccgctaaagtacgtgttgagggtctccaa

SEQ ID NO: 47 - DNA, 560 bp, Artificial Sequence
  artificial sequence (d2)
gctgcttagcctataccgtaatcggtgtgcgtgaacactagccaggtactgaatctaggatcgctgtggatctaaccagtccgctacgacaagagtttactaggaccgcctaaatcatcggcgcttaccgttaagaaacctgtccggcgacatatacagtgccattgcgcttgagaatcatgctgtgcgagagacatacacggttccgagttgacatctacgtgaagggcatctttcgatgctgacccgaagtttatctgggaagctacgtcatttgcctaccgctgcgactaatctttgcagacgacatgctatgagcttgctggaccacgaatcgttaccagtcatctgagacacttggcatacgcttgggcttgatacacctatggatgggatacactgatcggctgccgcataatttgctacgccttacagagaagtgcagtctaccggctgttaatactccggctttacacgagaagctactgagggccatttgacacaatcgcgtgagtttgctgatctgacatgggctgaaacatgagcctccgaactatcgt

SEQ ID NO: 48 - DNA, 560 bp, Artificial Sequence
  artificial sequence (d2)
tacgtgagatcggtccgatatgagctgtccacaatagccatagactaggagtcacccttcgagtggttctagcacatccagatgacacactaagtgccctgttcgggacttgtaaagcacgattccttggttaagacgcctcccagtcagtatcatggtcgtaaagttcgtccagtggtcaacgctcttcgtcaagcgataagttaaagccggtagctgctcaagcctgccatacggattagttcaaacgagcctgtcgtgtacgttctccgcacaatgtctaacaatggtacggtgcagatagcttccgcccaggttattaaggcaaattggcccatccattctgtcggtcggcaaacagttcctgaaattccgctgaggttgtaagacccggtctgaatagccagatcaatacgtcggtgctgatgagtgccatcacagtttctctaggatagcgcacgttcatgtcgcgtaacgcatctagcatttaggtgcaacggtactacgtccaccagtaggaagttcgcataaacggtcaccttagcctgagtagccgtcaa

SEQ ID NO: 49 - DNA, 560 bp, Artificial Sequence
  artificial sequence (d2)
atgtccaaccgaaactcgtgatcttagtgaccgcacggatctgtcattcgagaagcgtagagacttatgcctgggccttaacttgtgctcagtagcctcaagagaactgcctcctgtctattacgggtaaactcctggtgatccagagacgtagtgtcagaacagcctagatgtgttgccacgacctgtaaacggctttcttacgacgcaatgctgatggtgactggcgattaacgaaccgaatcatcctgtgtgcatcctacggtgtgccatttgaaccagagagtatcttcgaccacgatctgcaagggtgtcatgcttgacctagagtaccacgttcagttgcctcatagggcttagcagcgtattcatgcgacttgcgataacgatgtcctgtacggacgttccatagtccgacaaacccatgtatgtctgcgagaggttagccaagagtgcttactccacctagtgagatgtagcgacaacgactgtgagtgtacgactccttagggtatagcgttgccaaacttcccaaggtagggagcctttcccattacgaa

SEQ ID NO: 50 - DNA, 560 bp, Artificial Sequence
  artificial sequence (d2)
tccacagtatcatccgatggagcgattcgcatacgacagtcaatggctattggtcaggacctagcttccaagtcaagggaaggtttcaggatcgtcgcatcgtactttcctacgaagtgcctaaagggatcactctccgaacggtttgtatcagcgtgcagatgtacctgttacgccagaggaatgacattctacccgagggatcttacagtccgggatttgtgcaatcacagttgggctctaacgtcaagcgaggtgtatgtcccatgaataaggacggctttctcaggccaagaagtctacgcagaagttacccagctcgtttacggtgtccactcaaagtctagcatgttccggtgacctagttgatggcagtagcagtaccatgacaagaggcttccgattatccagacccagttgtgggctaatatgagcagcaccctagtatttcgcgcaatgccggttatatgaaggccacgtacaagtttctccgcgcatgtgtcagatagtatccggttccacagcataagtccgccagttggttcactaagttgccgaca

SEQ ID NO: 51 - DNA, 560 bp, Artificial Sequence
  artificial sequence (d2)
tattgacgaccgttgccagagagccatcacttggtttcgactataacgacagatccgtggcctcctaaagttgcgtatgcagtatcgagatgtaccctgcgaaccgagtgtactaacgtgtctgaggaatccattcccgtatcgggcacaacagtatgtgtcttccagatagagggcctttgctgacgaagtcctagactatcgcttagagacgcctacagaccagtaatcgtgaccttctacctgagatgccgtgaacataggtgctaatccgagagcatgtgtacgaactccgaaccttgccattaagggatgagcctactgaactaccgctgatcgtgcgagtatatcctgctgctaacgtaaactcctgagggctacagctaaacagcttggacctagtgtcatatcgccgttccaactgactccttgagagactgcgtaagatttccgccgacattgccaaacgctaattgccgatggtgtaaacgacccgcattccattggttgctaaagcctcgtaagaatccgggctgactatcatgtgagcttgacgctac

SEQ ID NO: 52 - DNA, 560 bp, Artificial Sequence
  artificial sequence (d2)
aggtcctcagaggctaatgtttcatgcaatgagatcccgcgtggacaccaccaagattctactgttgtcaagatacgggcgactcgacatggagctactattctatcagaagagccctgccaggcgttcaatcgcatttccatttaatggctgactcgcgcagacgaagtctcctagagttaagtcttacgagcaccgcttgtgtgagcacgatcatacgatactgactaaggcgtcaccgagtttcagaccctacgacatgactgtctttaggccagagtctactagaccgagctttggatgccaacctttccgaagtgagatttacccacagcgttcgtgtgttcgactaacccgcaaagtgttaccataggctggtcctatttcgcagtggctagagagcaatgttccaggatgtgctactacttgccgtgagctagacataccgatggctaagtggatacgttacaggcgcacgtagttctaaccggcttatacggataacctgacccgagcgttattcttatgccgcagagaggtttcttacccgaaggcactag

SEQ ID NO: 53 - DNA, 560 bp, Artificial Sequence
  artificial sequence (d2)
gtcacatgcaagctgtttccttctacatgacgagcctctgcgataggtgagtatcccactcattgatagctgccgcaagtcaggagaatacgtccgttagtaaactgtcccatgccgaagctcaagacctggaagtccttgataactggcacactctgagccaactgaacgtgtacgcattacaactccggtgttagcctgcttagctgaaccagcagtaattgttaggcgtcccaacgatccatgatccgcgtgaagaaatctttagcgcccataggcagtaaggtagcccgacatagtgtctattaggcccgaaatcccttagggagcccaatacatgatcttagccgagtcgtaggaacgtccatctcgaaagtcgtttgctagggcaatccaagtctcgatcccgataagttctggctaggttgacaaagcgtccagatccgacgagtaaatggtccctgttaatccgatagtcgcgcaccacggtgaatatagtccgatgacattgacctgtaccagaccgcgtctcaaattgacgaaagcgatgttcgtaaccg

SEQ ID NO: 54 - DNA, 560 bp, Artificial Sequence
  artificial sequence (d2)
ggtggaaagctcgtctcccaatgccattagcctcggcggagcgatagcagctcctctggaagcatcagtgcgtctgcccaaggcgttcctcgtcggtacaacgtagactgccgctacggacggtgtcaccagggatacactccatagcatccgggtcgcaaggtgtgcgtgccaactacccgacttctaacagggctggccgatactgcgggctcaagtgactcagatcctgaagggcgcaccacgtcgcggactacagtgttcacatgaagcgcggtcgtgcagcgcatggtccataccaactgcctagtacgcgggactggcgtcgaatcgactcgtccttcggaaacatgacggcgcggcctaagcgagaactctgctcgtgtccatcaacggctggcggcgatatgtcctgacctcagccatagtgcctacctcgggagcgttcaagcgatcctcggtcttaacgggcgaactcgggctcgaaagcgaatgcctccctaagctcttcggtggcggacgcggaatcatagctcagcgaactctcacggttgcaggcg

SEQ ID NO: 55 - DNA, 560 bp, Artificial Sequence
  artificial sequence (d2)
gtcgtgacacgcttcgacgattgagtcgccgcctacgactgacgatcttccgcctgtagctggatgtgcccgatccgtgaggacattcccacctggactgactcgcatggagactgccacggtgattcgcaacagcccgtagaggcttcgttcgaccacccgatgctgaaagctgctgcgctgatctgagacctcggagggcgtaaactggacacctgccactcggactgtgttcgcacgtcggcttcatagccactggcaaccgcgcttgtgtgcagacggaaccctttagtgcctggcgatgaccctactcccggtgaacggcaatgcaatgggcctggaactgtgacgctcccgtaccttcccttgagaggacctggcatctggacgcaactcctgggtgtgacctgtgagcaacgcctcctactgggtatagcccgcgcttagacgctgctagagccggagacatacgatccctgcgcttacacgcacgcgataggtgcgctcgataatctcggcccggtagtgcaacctgaccagcggtagaccttgatgacggc

SEQ ID NO: 56 - DNA, 180 bp, Artificial Sequence
  artificial sequence (e5)
ataagagctttgagcccacccgcatactgatttgactgccttaacttggtgaagccctcggacggaaacttgacatctcgttctatctgaatgagcgcggcacagcttgagtctacttggaattgcattagcaccggcctgccttacaacactgttgcgtattggactaactagcggcct

SEQ ID NO: 57 - DNA, 180 bp, Artificial Sequence
  artificial sequence (e5)
gtagttaggcaactctaggcggcaactgctcatcaactaggagtacagtcaatctgacggacgcgctactgcatacttagtcatctactggttccagagccacgggtcatcgtaaattgggtattccgaaatggcccacacgccgttcacgtttcaaatgattggcatctagggacacct

SEQ ID NO: 58 - DNA, 1596 bp, Artificial Sequence
  nucleic acid 1
tgattacgtccctgccctttgtacacaccgcccgtcgctaattgtcagtctagcgaatcattataccgaagaacatccgtttatgagaacgtgctaccaattaactgtactaagctgtccaaacttggtcatttagaggaactaaaagtcgtaacaaggtttccgtaggtgaacctgcggaaggatcattatcataagcagagcctttatcccatataagctattgtcacgaagtgtcactgtgaacgaatgttctctaaacttactacggcttcagatgtaacggattcagactactctattcataacggactacagattgcgtcaactacgatattctcttgagatcacgattagcaagtacctttgcagcttgaaattaaccagacctttccttggaatgcctatacagagatttatcataccaggagttctccagattacctagatgtcttaacgagatacaggacttacacgatgacttagtgtgttgtttgcatcaacctaacagtaactgagcgaattgtaccaacgtattctttaccggaagtaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttagttgtctgccagaaatcattgaacattccgacgaatatcgacatggttgcttatctaagaccttaaacggtacttggttagctgatcgcaatacttgaaagacttgatcctgtacttacctggacacgatgtaataatctcacacagttatgagaagctggttgcacctaaatagtcaattagcacgtagtaacgtagacttgccactgatgaaacatagtttgacctcaaatcaggtaggagtacccgctgaacttaagcatatcaataagcggaggaaaagaaaccaacgaacgattgaagatgtactcagatattcattgatgggcctacgtctacttactatgggaatgtaaatactctgttccagcctaaggttagctttgcgaatacaaatgttcttatcgacgcacagtcatacggattacgatcaagttaatggttactccctaccgattattgcatccagatcatattgagaggaatcacctgtacggtttagaaatcagctctactagaagacactattgccatacgtcaaattgcagtgagtttcaccaaatcatggagatgttacccagttagcatacaactctttgcacaagtgcataatgtagtccctatgtcacaaggttatacgaagcatgtcaaatcatcgcctttagttacgatgtagttccacaagcgaaattagtttccgaaatggtcaagcatccaagtttagctcgaatctttaaggagatactcgaagtgcctatattacggaggtattatcatgtagcaagcgttacctagcttattagtccacgaatcatgtgttagaagtcgtcaagttcatgttatcctaccagccgcccgtcttgaaacacggaccaaggagtctaac

SEQ ID NO: 59 - DNA, 1320 bp, Artificial Sequence
  nucleic acid 2
tgattacgtccctgccctttgtacacaccgcccgtcgctattactgatcgaacgtcgtataatgctgaggcatctgttattaaccgtacctttcaaggattaccatgtggcaacataagtaaacttggtcatttagaggaagtaaaagtcgtaacaaggtttccgtaggtgaacctgcggaaggatcacatccttggtctaagaaagtgcatgatttgagcataccaatcgccattacgataaagatcctttgagtctaacgtacactgtgtcatctgtaagataccattgtcactacttcagtcagaactttcaacaacggatctcttggcttccacatcgatgaagaacgcagcgaaatgcgataagtaatgtgaattgcagaattcagtgaatcatcgaatctttgaacgcaacttgcgccctttggtattccgaagggcatgcctgtttgagagcattgaacacttcgtaaggtacacctatggatcaacgattaagtctcgataccgtaagatggtaactctagtcagtgataatcaacagcgtagtacattcgtaagcagtcttggacattactttctgagtgcaacattcaacgtctaaacgggttaaatctctcataacggaacttgtgtgcaacagatgctatatggtatgcaaatgcgatacactttgacccgctgaacttaagcatatcaataagcggaggaaaagaaactaacgtaaagctattaaccggagtgaatccttcattaaagtcgcacaagctgtattaccgttacgcaacgtatttgattgaccatgtgaacagaagtaccctattgacctagattatgcagcaatgcctaagactatttgcctaattcgggctatttagaccaatcctccatgatgtatatcagtcaaggctagtttggaacatacacgaaagtccttatgtagtagagtgcaattctcgtatccttcaacagtgttatcgagtatcgaacgattatcctatgggtatccacttatagaacgtgtgtagactaacctgtaaacgatgtctctgaaagcaagactacttatctgagatcggatgtttaagacgctatgacaccattaacttatgccagtgctagtcattatgaccacgatttggaatttatggctatcgccactatgaaatgctaagctacctgaacaatttgtacgcagtgacagtagatcctttgatccagaacttattaagagctgaccctatgaaacgtgatgtcctattcattattacgggaaaccgtagcgacccgtcttgaaacacggaccaaggagtctaac

SEQ ID NO: 60 - DNA, 1919 bp, Artificial Sequence
  nucleic acid 3
tgattacgtccctgccctttgtacacaccgcccgtcgctattggccttcagtcgagaacttgttgaaactgtcctgacgcactggaacgagcttccattgattcgctagaaatgccgaccaaacttggtcatttagaggaactaaaagtcgtaacaaggtttccgtaggtgaacctgcggaaggatcattacacagtgtggatctgacgaattaccaaggcactccatgtgtgccatctacgtctcaggaattgtacctgctaccactaggcatcgagaacgctgcatgtattcaccgagtaaggtcttccagactccgataccgtatgtgttcccaggagaaatgtcgcttagccggttcaagccatcatgtgctagactagacacgtctatcgcggtttacacgaccatcagttgagccaatgctatccttgcgggtcaaacagagcttacggatcacccatagttgtcacgccacgttaaagttccgagcgaaacgctatctcttcgagagctgtcccaatgaaactctgcacggacttgtattgcacaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttactatgaggcccacagttacgaacgactagaccactgtcttacgagtgtcgcaccataagatggcgagtaatccgctcaatccactggttcctgagaaagagccggaaatctgaggtcattctgcccatgatagctggaaacacccgagtctctaagtgtgagtagcctgatctactgcaaacgcccgatacatatcgtgagagtctgctaggactgatcgtttgacctcaaatcaggtaggagtacccgctgaacttaagcatatcaataagcggaggaaaagaaaccaactcaggctatattgaggcaccgcctggctagtagattacgacagctataacttcgggcaagccggttgatccaactatcgaaacctcgttagagcagtgtgtggcctaatggcatactggaacctatctgttacgccgagaactcgtgagcaactcagtctcataaagtcatggtccgcactgatgctgcacaaagctaccgattgatacgttcgccgactgtgatgcgtgaatcattccgtcaaagtgtccacccgtgtaggcattggtatatcgaccgatccaagaagcgacgcttagtacgcgattacattgggcagatggtacagctcccataaacgctaggaactgttcgcaagagtcctgtgtcagagtcaaggataccgttcagaggcaaactgaccgtcattcgtgctaaacgatgtgatccgccctttcagacgctagtgttacctggaagaagattggcgctacctatgtcccatacagcgacaaggtcttgtagaaggcatgtcaagctccctaaatggctccgctaaagtacgtgttgagggtctccaaccgcccgtcttgaaacacggaccaaggagtctaacaaacaccggctaactccgtgccagcagccgcggtaatacggagggtgcaagcgttaatcggaattactgggcgtaaagcgcacgcaggcggttataagagctttgagcccacccgcatactgatttgactgccttaacttggtgaagccctcggacggaaacttgacatctcgttctatctgaatgagcgcggcacagcttgagtctacttggaattgcattagcaccggcctgccttacaacactgttgcgtattggactaactagcggcctgtggggagcaaacaggattagataccctggtagtccacgccgtaaacgat

SEQ ID NO: 61 - DNA, 1919 bp, Artificial Sequence
  nucleic acid 4
tgattacgtccctgccctttgtacacaccgcccgtcgctacctagaaagctcgccattagccgcagtagtgattggacatcagagtttcgctcacaacgtcaccgctcgttatggaacttaaacttggtcatttagaggaactaaaagtcgtaacaaggtttccgtaggtgaacctgcggaaggatcattaaagcgttggttcgttacgcaaggctctacgaaagcagtgtctacttagcgttcagtgcagcgatccacaatctcatgggtatgtcatcgaccagctacgacgcaagtttcccagatcaagattaggtgcccttcaagcacggttggaactctaccgacaattacgaggtcccaattacgggtggcaactatgctgtaccagtaagatcctgccgattcgacgcacagtcataactcagtgtacgtgtatcctggcaaggaggaagctccctttacatgctagtgcaatgtccgcagtttgcgagaggactatatccagtctaccacaggtcagaggttacaccctggctatctagtatggaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttaccgtaaagctaggtcaggtcttcactgggcaacgacataatgggtaactcacttccagcctacatcagcggtgtcaaaggtagatgcctatcgtaccacccacaatgctctagggtttcagagaagctgtgtcttccgatggtcaccagatggattcgactcaaggtcatacaggagtgtcgcgtaacatagcctatgcaaccgttcggttaaggacgtgtttgacctcaaatcaggtaggagtacccgctgaacttaagcatatcaataagcggaggaaaagaaaccaacgctgcttagcctataccgtaatcggtgtgcgtgaacactagccaggtactgaatctaggatcgctgtggatctaaccagtccgctacgacaagagtttactaggaccgcctaaatcatcggcgcttaccgttaagaaacctgtccggcgacatatacagtgccattgcgcttgagaatcatgctgtgcgagagacatacacggttccgagttgacatctacgtgaagggcatctttcgatgctgacccgaagtttatctgggaagctacgtcatttgcctaccgctgcgactaatctttgcagacgacatgctatgagcttgctggaccacgaatcgttaccagtcatctgagacacttggcatacgcttgggcttgatacacctatggatgggatacactgatcggctgccgcataatttgctacgccttacagagaagtgcagtctaccggctgttaatactccggctttacacgagaagctactgagggccatttgacacaatcgcgtgagtttgctgatctgacatgggctgaaacatgagcctccgaactatcgtccgcccgtcttgaaacacggaccaaggagtctaacaaacaccggctaactccgtgccagcagccgcggtaatacggagggtgcaagcgttaatcggaattactgggcgtaaagcgcacgcaggcggttgtagttaggcaactctaggcggcaactgctcatcaactaggagtacagtcaatctgacggacgcgctactgcatacttagtcatctactggttccagagccacgggtcatcgtaaattgggtattccgaaatggcccacacgccgttcacgtttcaaatgattggcatctagggacacctgtggggagcaaacaggattagataccctggtagtccacgccgtaaacgat

SEQ ID NO: 62 - DNA, 1596 bp, Artificial Sequence
  nucleic acid 5
tgattacgtccctgccctttgtacacaccgcccgtcgctatcaggaagtgtgtcccattgccggaggagtcctattgaatcacggattacgtctgtaacgctggaccgaggttgtatcataaacttggtcatttagaggaactaaaagtcgtaacaaggtttccgtaggtgaacctgcggaaggatcattagcttcgattacgatgcccaaatacgatccgcgtagtttccacgaggtctacagtaccctattgttcgaggcagtaacctgaaccgcgtctgtcaacagttatgtgacggcaagttgtccaagtccgagccatactatcagtcgtcttagctcatgggaagctcgcagtgttaagctcagtaggcaaattccagcgtgatgccgatccagtgtacgagaatccttacatgcaagtgtcgcaggccagatcagtttcgagaaagagtacgttctatccctggcgtcctcagtgactcaagatgagattacatccacacggtctcggtccattcgcaaagtacagtgtttccttagcagcaggaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttaacatgctgcgtagtacgtcgatcaccaagctatgagcgttgtcaaaggagtgtcaaccgacgagtccaggtttcatcaccttgctaggtatccacaggtgcattaggcggctaagtcttccacatcgtattgccgaagtgtatcgcccagacattcaagctgtcagaactctgcgttacagaacgtgccgtcaagattcaggctatcatccgtgaaccagtttgacctcaaatcaggtaggagtacccgctgaacttaagcatatcaataagcggaggaaaagaaaccaactacgtgagatcggtccgatatgagctgtccacaatagccatagactaggagtcacccttcgagtggttctagcacatccagatgacacactaagtgccctgttcgggacttgtaaagcacgattccttggttaagacgcctcccagtcagtatcatggtcgtaaagttcgtccagtggtcaacgctcttcgtcaagcgataagttaaagccggtagctgctcaagcctgccatacggattagttcaaacgagcctgtcgtgtacgttctccgcacaatgtctaacaatggtacggtgcagatagcttccgcccaggttattaaggcaaattggcccatccattctgtcggtcggcaaacagttcctgaaattccgctgaggttgtaagacccggtctgaatagccagatcaatacgtcggtgctgatgagtgccatcacagtttctctaggatagcgcacgttcatgtcgcgtaacgcatctagcatttaggtgcaacggtactacgtccaccagtaggaagttcgcataaacggtcaccttagcctgagtagccgtcaaccgcccgtcttgaaacacggaccaaggagtctaac

SEQ ID NO: 63 - DNA, 1596 bp, Artificial Sequence
  nucleic acid 6
tgattacgtccctgccctttgtacacaccgcccgtcgctatcccgcaaatacctttggagtgcgtcactatctaggagtgtgccgatgactcgtaatctccatcctcgaagttgcacgataaacttggtcatttagaggaactaaaagtcgtaacaaggtttccgtaggtgaacctgcggaaggatcattaataatccagggtccacgagtgaatgccctgcaaatgtaccaagttcctgaccttctggcatgtgaagccgatcttatcgctgaagagtctcgaagtcgctgacatacacccgtattgtcgatctgttggcgtaacggacatacgatgcactgacagcagttgcttagagcctagacacgacattgccttgaacgaccttgctactcatagggatacccgacgtagacgtttagtcctgcaagtcgaaagccctttgtgagagtcgccttatagtaccggatagtctcccagccatattggagagtccatatagccacggtagaatgctccgaggtaacctgagtcaaattgccgcactagaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttagtgacagttcacggtagcagctaaatcttcgggcatcacgagtacatgagtctcccatcgttaatccagcaagccgatgtggagctatttcaacgggacgtatatgtcgtccatccgagttgcggactatctacagggtgaattatgcgactgactgccttgccactacgaaacagtgcgttcaaattgcgctaagggcgtgcgaatacttatgcaggcgtttgacctcaaatcaggtaggagtacccgctgaacttaagcatatcaataagcggaggaaaagaaaccaacatgtccaaccgaaactcgtgatcttagtgaccgcacggatctgtcattcgagaagcgtagagacttatgcctgggccttaacttgtgctcagtagcctcaagagaactgcctcctgtctattacgggtaaactcctggtgatccagagacgtagtgtcagaacagcctagatgtgttgccacgacctgtaaacggctttcttacgacgcaatgctgatggtgactggcgattaacgaaccgaatcatcctgtgtgcatcctacggtgtgccatttgaaccagagagtatcttcgaccacgatctgcaagggtgtcatgcttgacctagagtaccacgttcagttgcctcatagggcttagcagcgtattcatgcgacttgcgataacgatgtcctgtacggacgttccatagtccgacaaacccatgtatgtctgcgagaggttagccaagagtgcttactccacctagtgagatgtagcgacaacgactgtgagtgtacgactccttagggtatagcgttgccaaacttcccaaggtagggagcctttcccattacgaaccgcccgtcttgaaacacggaccaaggagtctaac

SEQ ID NO: 64 - DNA, 1596 bp, Artificial Sequence
  nucleic acid 7
tgattacgtccctgccctttgtacacaccgcccgtcgctagacaccctgttcagattagcgagcctcagttacaccagattccgagttcgtaagatcgagaggagccatcatggacgtttaaacttggtcatttagaggaactaaaagtcgtaacaaggtttccgtaggtgaacctgcggaaggatcattactgacggaccaatctgtatgtaaagcggctattcaggagcctatccgacgagttgatgcttacaaggcgatctatccctgaccagtgctaaccatgtgcataagagcagtctcactcacgagtctcggttccttagacgattcaatgccaagttgtgccggagaacacctgttgatcctcgacaatgattcagtccaccgggatgtctgtagttcccaacgccaatatgtagagcttcggtccacgaaagtaccgtggtagccatgatatgacttacgcccgacaaagttcgggagtttctcgcatgtgaagtttccgcaaccatgagcaaggtcgtttgacctggaagtgtatgatccgaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttatctgacagccttctacgagcctgctgaatcagatgaaccacttggtcgcaatgatcgcaaggtcgggtatatcttcacggttagatccgaactgctccactgggtacaacacactgacttggtaactcggtcatacacgtcgggaacataactgcctgtgatagcacgcactcttaggacagtcgcattctctaggtcatggaatagcgcaacatcgctgtttgacctcaaatcaggtaggagtacccgctgaacttaagcatatcaataagcggaggaaaagaaaccaactccacagtatcatccgatggagcgattcgcatacgacagtcaatggctattggtcaggacctagcttccaagtcaagggaaggtttcaggatcgtcgcatcgtactttcctacgaagtgcctaaagggatcactctccgaacggtttgtatcagcgtgcagatgtacctgttacgccagaggaatgacattctacccgagggatcttacagtccgggatttgtgcaatcacagttgggctctaacgtcaagcgaggtgtatgtcccatgaataaggacggctttctcaggccaagaagtctacgcagaagttacccagctcgtttacggtgtccactcaaagtctagcatgttccggtgacctagttgatggcagtagcagtaccatgacaagaggcttccgattatccagacccagttgtgggctaatatgagcagcaccctagtatttcgcgcaatgccggttatatgaaggccacgtacaagtttctccgcgcatgtgtcagatagtatccggttccacagcataagtccgccagttggttcactaagttgccgacaccgcccgtcttgaaacacggaccaaggagtctaac

SEQ ID NO: 65 - DNA, 1596 bp, Artificial Sequence
  nucleic acid 8
tgattacgtccctgccctttgtacacaccgcccgtcgctacatgactggaaaccctctgacgtgtaactctggaagctcagttatcggaaacggcgctaagctacgtgatcgtaagcagtaaacttggtcatttagaggaactaaaagtcgtaacaaggtttccgtaggtgaacctgcggaaggatcattactctgatggacctggtgatacacggtactatttggcatggtcacatcgggcatctgtaagacctccagttgtagtgtgcagagttcccagacagtctaagacggcattgactatggccttgtggttcgagaaccgaacatccaagagtttcgctcgttcatggcgataacccttcaacgtgtggtaacctgtaacgcagtcagctttagcgcgtgaataccttgaggcaatacaccgagttgtgctaccctagtgatgacagaatggcaccttatgctccggtacacctacggaatcatgcaagtggaatccctttcgagagcaggctcagtttagttgcgaagtgatctccgcatttccaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttaacttagggagtatgccgtcgaacatcgctcgtgagtaacttatcgtgcggatacacctcgtacatgccactcggtacttagaatagctggtaacctccgatgctcgcaatgcgtagttctggattccaatggaccaacggtcattcctgggtgacaaagcaatctcctgtagcaggtcacagttctcgtctcgcagtaacgaagtcctcttacgtcatggtttgacctcaaatcaggtaggagtacccgctgaacttaagcatatcaataagcggaggaaaagaaaccaactattgacgaccgttgccagagagccatcacttggtttcgactataacgacagatccgtggcctcctaaagttgcgtatgcagtatcgagatgtaccctgcgaaccgagtgtactaacgtgtctgaggaatccattcccgtatcgggcacaacagtatgtgtcttccagatagagggcctttgctgacgaagtcctagactatcgcttagagacgcctacagaccagtaatcgtgaccttctacctgagatgccgtgaacataggtgctaatccgagagcatgtgtacgaactccgaaccttgccattaagggatgagcctactgaactaccgctgatcgtgcgagtatatcctgctgctaacgtaaactcctgagggctacagctaaacagcttggacctagtgtcatatcgccgttccaactgactccttgagagactgcgtaagatttccgccgacattgccaaacgctaattgccgatggtgtaaacgacccgcattccattggttgctaaagcctcgtaagaatccgggctgactatcatgtgagcttgacgctacccgcccgtcttgaaacacggaccaaggagtctaac

SEQ ID NO: 66 - DNA, 1320 bp, Artificial Sequence
  nucleic acid 9
tgattacgtccctgccctttgtacacaccgcccgtcgctagcacctagcctttaacgagaagaatgtagccctacgccatcggcatgtgattccatacgatgttacgaaacctgaggcagaaacttggtcatttagaggaagtaaaagtcgtaacaaggtttccgtaggtgaacctgcggaaggatcacttctgaaactatgacgcgccaaccggaatcgtgtaatggattgacctacttgctcggacgacggataacgctgtatgcaaatgtgcctgtaactcggctctgcgaactgctctgatctaactttcaacaacggatctcttggcttccacatcgatgaagaacgcagcgaaatgcgataagtaatgtgaattgcagaattcagtgaatcatcgaatctttgaacgcaacttgcgccctttggtattccgaagggcatgcctgtttgagagtccacgtaaatcagcgcgttatgggtctgacgtaagcacaagggtcctatacacgctactctggttatccctgagaagtcggttaccatgtcacacagtcaggctatatgccctcacgttgattcgagcgaagttactgcaccaagtctggcgtagttagtgttccgtagagcaagtcactcaatcccgagcaaagtgtcgtgatgctgttcagcaagacacccgctgaacttaagcatatcaataagcggaggaaaagaaactaacaggtcctcagaggctaatgtttcatgcaatgagatcccgcgtggacaccaccaagattctactgttgtcaagatacgggcgactcgacatggagctactattctatcagaagagccctgccaggcgttcaatcgcatttccatttaatggctgactcgcgcagacgaagtctcctagagttaagtcttacgagcaccgcttgtgtgagcacgatcatacgatactgactaaggcgtcaccgagtttcagaccctacgacatgactgtctttaggccagagtctactagaccgagctttggatgccaacctttccgaagtgagatttacccacagcgttcgtgtgttcgactaacccgcaaagtgttaccataggctggtcctatttcgcagtggctagagagcaatgttccaggatgtgctactacttgccgtgagctagacataccgatggctaagtggatacgttacaggcgcacgtagttctaaccggcttatacggataacctgacccgagcgttattcttatgccgcagagaggtttcttacccgaaggcactagcgacccgtcttgaaacacggaccaaggagtctaac

SEQ ID NO: 67 - DNA, 1320 bp, Artificial Sequence
  nucleic acid 10
tgattacgtccctgccctttgtacacaccgcccgtcgctatgcggagcatcctagtacaatatccggttgcctataagcccggtatgcgcgaattaacctaactgccagagatgagttccaaacttggtcatttagaggaagtaaaagtcgtaacaaggtttccgtaggtgaacctgcggaaggatcataggtcacgctagtaccaaggagactcagaccttacagcttgcttgcagacagatcggaatcccacagcagagtttagacgtttggagacagtcccacttcagtcgttggatgcacttagactttcaacaacggatctcttggcttccacatcgatgaagaacgcagcgaaatgcgataagtaatgtgaattgcagaattcagtgaatcatcgaatctttgaacgcaacttgcgccctttggtattccgaagggcatgcctgtttgagagcagggttccctagtaagtacgattccaatacgcgatccgaatgcggcgtttcctaagcaaggtataatctcctgacgaggagtcgggtccataaggtttccatagttcaccgtgagactgcgatggtctgccaatgttcacttcaagtccgtaagacacggcaagagcctagcatctgttcgttcagagtcatggtatcggacaactgcctgatcttcgaacccgctgaacttaagcatatcaataagcggaggaaaagaaactaacgtcacatgcaagctgtttccttctacatgacgagcctctgcgataggtgagtatcccactcattgatagctgccgcaagtcaggagaatacgtccgttagtaaactgtcccatgccgaagctcaagacctggaagtccttgataactggcacactctgagccaactgaacgtgtacgcattacaactccggtgttagcctgcttagctgaaccagcagtaattgttaggcgtcccaacgatccatgatccgcgtgaagaaatctttagcgcccataggcagtaaggtagcccgacatagtgtctattaggcccgaaatcccttagggagcccaatacatgatcttagccgagtcgtaggaacgtccatctcgaaagtcgtttgctagggcaatccaagtctcgatcccgataagttctggctaggttgacaaagcgtccagatccgacgagtaaatggtccctgttaatccgatagtcgcgcaccacggtgaatatagtccgatgacattgacctgtaccagaccgcgtctcaaattgacgaaagcgatgttcgtaaccgcgacccgtcttgaaacacggaccaaggagtctaac

SEQ ID NO: 68 - DNA, 1596 bp, Artificial Sequence
  nucleic acid 11
tgattacgtccctgccctttgtacacaccgcccgtcgctaacggcactgatgttcacccgccgtcgatcatacacgcagggcgatgactctatgcgaggctccgaccagtaacaggcgctaaacttggtcatttagaggaactaaaagtcgtaacaaggtttccgtaggtgaacctgcggaaggatcattacctggcgaatgtctaaggcgtccatatccgaggtgcagcgcgttgcctgaccattaggcccgtatagttcggcgtgaccgagatgccgctcagtacgacggtctaacaagctggccgcacttgccaacctgtcgcggactgtcttaacggtggcccgacttgctaccacacccgtgggattgtgctacgaagcgtcccgaaggtcctcagcccaagagtcctgtagtgagtacccggagcctcgaccctgatgtgatccgaccagattggagccggtgaccctcagacggagtcaaggtcctacctgtgaagccctgacggcgtggattcctgctagagccaaggagagtgtcccgctacaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttgcggacgatgcctttgtcgataatgctcccgctgtaggccagcgccaatcggctgtgcatttagcgaggtctcacgccagtgcgagtacgagccttcctcctaagcgttcggtcggacaggacatctggatcgcggaaccctaatcccgtgggacaccgtcacttggtcgatgcgcgtagcttgtcaccgcagggactgagaggtcaacccatgcgactggtttgacctcaaatcaggtaggagtacccgctgaacttaagcatatcaataagcggaggaaaagaaaccaacggtggaaagctcgtctcccaatgccattagcctcggcggagcgatagcagctcctctggaagcatcagtgcgtctgcccaaggcgttcctcgtcggtacaacgtagactgccgctacggacggtgtcaccagggatacactccatagcatccgggtcgcaaggtgtgcgtgccaactacccgacttctaacagggctggccgatactgcgggctcaagtgactcagatcctgaagggcgcaccacgtcgcggactacagtgttcacatgaagcgcggtcgtgcagcgcatggtccataccaactgcctagtacgcgggactggcgtcgaatcgactcgtccttcggaaacatgacggcgcggcctaagcgagaactctgctcgtgtccatcaacggctggcggcgatatgtcctgacctcagccatagtgcctacctcgggagcgttcaagcgatcctcggtcttaacgggcgaactcgggctcgaaagcgaatgcctccctaagctcttcggtggcggacgcggaatcatagctcagcgaactctcacggttgcaggcgccgcccgtcttgaaacacggaccaaggagtctaac

SEQ ID NO: 69 - DNA, 1320 bp, Artificial Sequence
  nucleic acid 12
tgattacgtccctgccctttgtacacaccgcccgtcgctacgtacctgtcagcacgctgttgaccttagcccgtggcaacgactgtgaagcctccgacacgtactgagggcgattcccagaaacttggtcatttagaggaagtaaaagtcgtaacaaggtttccgtaggtgaacctgcggaaggatcaccatactgcgaatgggagccgccggaggtaagtcctttccctgatgaccttgcgcgtagggccgggtaagagcttctccactgactgtcaaccgtgggcacgccgaggatgctactcatgactttcaacaacggatctcttggcttccacatcgatgaagaacgcagcgaaatgcgataagtaatgtgaattgcagaattcagtgaatcatcgaatctttgaacgcaacttgcgccctttggtattccgaagggcatgcctgtttgagagggcagctttacggttcccagtgcctaatgaggacgcctgggcggaatcgagccttcggaaagacatctgcagcacggtgcctgcaacctgtcggtgacgtatcaggacctggtgtccacccgttgtcagggcttccaaggtcaagcaagtggtgaccggccatgcgtggtcgcttcacagaacatcacggcagtcgccgtatcggcccgagtgagactagacccgctgaacttaagcatatcaataagcggaggaaaagaaactaacgtcgtgacacgcttcgacgattgagtcgccgcctacgactgacgatcttccgcctgtagctggatgtgcccgatccgtgaggacattcccacctggactgactcgcatggagactgccacggtgattcgcaacagcccgtagaggcttcgttcgaccacccgatgctgaaagctgctgcgctgatctgagacctcggagggcgtaaactggacacctgccactcggactgtgttcgcacgtcggcttcatagccactggcaaccgcgcttgtgtgcagacggaaccctttagtgcctggcgatgaccctactcccggtgaacggcaatgcaatgggcctggaactgtgacgctcccgtaccttcccttgagaggacctggcatctggacgcaactcctgggtgtgacctgtgagcaacgcctcctactgggtatagcccgcgcttagacgctgctagagccggagacatacgatccctgcgcttacacgcacgcgataggtgcgctcgataatctcggcccggtagtgcaacctgaccagcggtagaccttgatgacggccgacccgtcttgaaacacggaccaaggagtctaac

SEQ ID NO: 70 - DNA, 409 bp, Artificial Sequence
  nucleic acid 13
aactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttgtcgggcgactgctctcatgaccagcgtgggcgtccatggctgagcctcgtgtggctcgagccgacgtctggccgtgagctcgggagggctggtcgagctgctgccacgctctcggctcgatcaccgtgtgacgtcggcgactccaccacggcacggcgacggtgtcacgcgctcctggggtttgacctcaaatcaggtaggagtacccgctgaacttaagcatatcaataagcggaggaaaagaaaccaac

SEQ ID NO: 71 - DNA, 409 bp, Artificial Sequence
  nucleic acid 14
aactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttcccaggagcgcgtgacaccgtcgccgtgccgtggtggagtcgccgacgtcacacggtgatcgagccgagagcgtggcagcatttatattgcaatataaatgctgccacgctctcggctcgatcaccgtgtgacgtcggcgactccaccacggcacggcgacggtgtcacgcgctcctggggtttgacctcaaatcaggtaggagtacccgctgaacttaagcatatcaataagcggaggaaaagaaaccaac

SEQ ID NO: 72 - DNA, 409 bp, Artificial Sequence
  nucleic acid 15
aactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcattttaaggcccatgttgtaggtcgaattgctagcaattcgacctacaacatgggccttaatgctgtgcgcaccaagaggatcaaccagtgtcggatgcatccgacactggttgatcctcttggtgcgcacagcatttacccagaagtgtattcctcgaggaatacacttctgggtaagcgtaggtttgacctcaaatcaggtaggagtacccgctgaacttaagcatatcaataagcggaggaaaagaaaccaac

SEQ ID NO: 73 - DNA, 409 bp, Artificial Sequence
  nucleic acid 16
aactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttgtggtggagtcgccgacgtcacacggtgatcgagccgagagcgtggcagcatttatattgcaatataaatgctgccacgctctcggctcgatcaccgtgtgacgtcggcgactccaccacggcacggcgacggtgtcacgcgctcctgggttaccgcggctagttcggcgtggctggcacgtttgacctcaaatcaggtaggagtacccgctgaacttaagcatatcaataagcggaggaaaagaaaccaac

SEQ ID NO: 74 - DNA, 409 bp, Artificial Sequence
  nucleic acid 17
aactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttggggcggttaaggaaagtcaaactcccgggctgtgaaggcccagtaggttgcgtagctaagacagcacctcataggcatgctgtgcgcaccaagaggatcatgcctatgaggtgctgtcttagctacgcaacctactgggcctaccaagagacgttacccgttaccgcggcggctggcacgtttgacctcaaatcaggtaggagtacccgctgaacttaagcatatcaataagcggaggaaaagaaaccaac

SEQ ID NO: 75 - DNA, 16 bp, Artificial Sequence
  18SV9f
gtacacaccgcccgtc

SEQ ID NO: 76 - DNA, 23 bp, Artificial Sequence
  18SV9r
gatccttcygcaggttcacctac

SEQ ID NO: 77 - DNA, 22 bp, Artificial Sequence
  ITS1f
cttgrtcatttagaggaastaa

SEQ ID NO: 78 - DNA, 20 bp, Artificial Sequence
  ITS1r
gctgcgttcttcatcgwtgy

SEQ ID NO: 79 - DNA, 20 bp, Artificial Sequence
  ITS2f
rcawcgatgaagaacgcagc

SEQ ID NO: 80 - DNA, 20 bp, Artificial Sequence
  ITS2r
tcctccgcttattgatatgc

SEQ ID NO: 81 - DNA, 17 bp, Artificial Sequence
  LR0f
acccgctgaacttaagc

SEQ ID NO: 82 - DNA, 19 bp, Artificial Sequence
  LR3r
ggtccgtgtttcaagacgg

SEQ ID NO: 83 - DNA, 19 bp, Artificial Sequence
  U515
gtgycagcmgccgcggtaa

SEQ ID NO: 84 - DNA, 20 bp, Artificial Sequence
  U806
  misc_feature (8)..(8): n is a, c, g, or t
ggactacnvgggtwtctaat

(19) United States
(12) Patent Application Publication
(10) Pub. No.: US 2024/0287604 A1
(43) Pub. Date: Aug. 29, 2024

(54) INTERNAL STANDARD NUCLEIC ACID FOR QUANTIFYING EUKARYOTIC MICROORGANISMS

(71) Applicant: NATIONAL INSTITUTE OF ADVANCED INDUSTRIAL SCIENCE AND TECHNOLOGY, Chiyoda-ku, Tokyo (JP)

(72) Inventors: Dieter Tourlousse, Tsukuba-shi Ibaraki (JP); Yuji Sekiguchi, Tsukuba-shi Ibaraki (JP)

(21) Appl. No.: 18/568,042

(22) PCT Filed: Jun. 6, 2022
(86) PCT No.: PCT/JP2022/022726

§ 371 Date: Dec. 7, 2023

(30) Foreign Application Priority Data
Jun. 8, 2021 (JP) 2021-096217

(51) Int. Cl. C12Q 1/6876; C12N 5/10; C12N 15/11; C12N 15/63

(52) U.S. Cl. CPC C12Q 1/6876; C12N 5/10; C12N 15/11; C12N 15/63

(57) ABSTRACT

A nucleic acid comprising a partial nucleic acid sequence and/or at least one complementary sequence thereof, the partial nucleic acid sequence consisting of: (1) a 5′ flanking sequence comprising a nucleic acid sequence derived from a eukaryotic rRNA-related gene; (2) an artificial nucleic acid sequence consisting of a non-naturally occurring nucleic acid sequence; and (3) a 3′ flanking sequence comprising a nucleic acid sequence derived from a eukaryotic rRNA-related gene.

### TECHNICAL FIELD

The present invention relates to a nucleic acid as an internal standard for quantifying eukaryotic microorganisms.

### BACKGROUND ART

A variety of microorganisms live in all types of environments including natural environments such as soil and the ocean, intestines of animals, and human dwelling spaces such as houses. In many cases, microorganisms colonize each environment with a unique composition, and this collection of microorganisms is called a microbiota. In recent microbiome analysis, metagenome analysis methods based on phylogenetic classification are widely performed using the 16S ribosomal RNA (rRNA) genes as indices for prokaryotes, or the 18S rRNA genes, the ITS (Internal Transcribed Spacer) region, and the 25-28S rRNA gene sequence as indices for eukaryotes. In these methods, the types of microorganisms constituting microbiota are comprehensively identified by amplifying all rRNA-related genes contained in a sample by PCR using universal primers designed for highly conserved sequence regions of the rRNA-related genes. Next-generation sequencers can not only comprehensively sequence amplified rRNA-related genes, but also count amplified products at the molecular level, thus obtaining not only the types of microorganisms constituting the microbiota, but also the relative values of the abundances thereof (Non-Patent Document 1). However, since bias is inevitable in the series of processes for extracting nucleic acids from samples and amplifying them by PCR, the relative values of the abundance based on the counts of the amplified products do not accurately indicate the abundance ratios of microorganisms constituting the microbiota. Accordingly, an accuracy control method is required to accurately identify and correct such biases.

To control the accuracy of PCR, a method to correct the measured value using an exogenous nucleic acid having a sequence that is not present in the sample (spike-in control) as an internal standard is already known, and standard nucleic acids consisting of non-natural nucleic acid sequences have been developed (Patent Document 1). However, standard nucleic acids consisting of non-natural nucleic acid sequences cannot be amplified using universal primers for rRNA-related genes, and primers different from the universal primers must be used to amplify the standard nucleic acids. In that case, the amplification efficiency of standard nucleic acids cannot be considered to be equivalent to the amplification efficiency of rRNA-related genes, and strict accuracy control remains difficult.

Furthermore, when it is desired to simultaneously analyze prokaryotic microorganisms and eukaryotic microorganisms contained in a microbiota, a similar problem exists because the primers for the respective rRNA-related genes are different.

### CITATION LIST

### Patent Document

[Patent Document 1] JP 5229895 B

### Non-Patent Document

[Non-Patent Document 1] Francesca De Filippis, et al., 2017, Applied and Environmental Microbiology, Vol. 83, e00905-17

### SUMMARY OF INVENTION

### Technical Problem

The present invention has been made for the purpose of providing an internal standard nucleic acid optimized for accuracy control of detection and quantification of eukaryotic and/or prokaryotic microorganisms constituting a microbiota.

### Solution to Problem

The inventors have already developed internal standard nucleic acids optimized for accuracy control of detection and quantification of prokaryotic microorganisms (JP 6479336 B). Subsequently, the inventors have succeeded in producing internal standard nucleic acids for accuracy control of detection and quantification of eukaryotic microorganisms, and have completed the present invention.

Specifically, according to one embodiment, the present invention provides a nucleic acid comprising at least one partial nucleic acid sequence and/or a complementary sequence thereof, the partial nucleic acid sequence consisting of: (1) a 5′ flanking sequence comprising a nucleic acid sequence derived from a eukaryotic rRNA-related gene; (2) an artificial nucleic acid sequence consisting of a non-naturally occurring nucleic acid sequence; and (3) a 3′ flanking sequence comprising a nucleic acid sequence derived from a eukaryotic rRNA-related gene, wherein the partial nucleic acid sequence is selected from the group consisting of partial nucleic acid sequences (a) to (d):
a partial nucleic acid sequence (a) consisting of:
   (a1) a 5′ flanking sequence comprising at least 20 continuous nucleotides in the nucleic acid sequence of SEQ ID NO: 1;
   (a2) an artificial nucleic acid sequence consisting of any one of the nucleic acid sequences of SEQ ID NOs: 8 to 19; and
   (a3) a 3′ flanking sequence comprising at least 20 continuous nucleotides in the nucleic acid sequence of SEQ ID NO: 2;
a partial nucleic acid sequence (b) consisting of:
   (b1) a 5′ flanking sequence comprising at least 20 continuous nucleotides in the nucleic acid sequence of SEQ ID NO: 2;
   (b2) an artificial nucleic acid sequence selected from the group consisting of SEQ ID NOs: 20 to 31; and
   (b3) a 3′ flanking sequence comprising at least 20 continuous nucleotides in the nucleic acid sequence of SEQ ID NO: 3;
a partial nucleic acid sequence (c) consisting of:
   (c1) a 5′ flanking sequence comprising at least 20 continuous nucleotides in the nucleic acid sequence of SEQ ID NO: 3;
   (c2) an artificial nucleic acid sequence selected from the group consisting of SEQ ID NO: 32 to 43; and
   (c3) a 3′ flanking sequence comprising at least 20 continuous nucleotides in the nucleic acid sequence of SEQ ID NO: 4; and
a partial nucleic acid sequence (d) consisting of:
   (d1) a 5′ flanking sequence comprising at least 20 continuous nucleotides in the nucleic acid sequence of SEQ ID NO: 4;
   (d2) an artificial nucleic acid sequence selected from the group consisting of SEQ ID NOs: 44 to 55; and
   (d3) a 3′ flanking sequence comprising at least 20 continuous nucleotides in the nucleic acid sequence of SEQ ID NO: 5.

In the nucleic acid, it is preferable that the partial nucleic acid sequence (a) consist of: (a1′) a 5′ flanking sequence comprising the nucleic acid sequence of SEQ ID NO: 1; (a2) an artificial nucleic acid sequence consisting of any one of the nucleic acid sequences of SEQ ID NOs: 8 to 19, and (a3′) a 3′ flanking sequence comprising the nucleic acid sequence of SEQ ID NO: 2; the partial nucleic acid sequence (b) consist of: (b1′) a 5′ flanking sequence comprising the nucleic acid sequence of SEQ ID NO: 2; (b2) an artificial nucleic acid sequence selected from the group consisting of SEQ ID NOs: 20 to 31; and (b3′) a 3′ flanking sequence comprising the nucleic acid sequence of SEQ ID NO: 3; the partial nucleic acid sequence (c) consist of: (c1′) a 5′ flanking sequence comprising the nucleic acid sequence of SEQ ID NO: 3; (c2) an artificial nucleic acid sequence selected from the group consisting of SEQ ID NO: 32 to 43; and (c3′) a 3′ flanking sequence comprising the nucleic acid sequence of SEQ ID NO: 4; and/or the partial nucleic acid sequence (d) consist of: (d1′) a 5′ flanking sequence comprising the nucleic acid sequence of SEQ ID NO: 4; (d2) an artificial nucleic acid sequence selected from the group consisting of SEQ ID NOs: 44 to 55; and (d3′) a 3′ flanking sequence comprising the nucleic acid sequence of SEQ ID NO: 5.

The nucleic acid preferably further comprises an additional partial nucleic acid sequence (e) and/or a complementary sequence thereof, the additional partial nucleic acid sequence (e) consisting of: (e4) a 5′ flanking sequence comprising a nucleic acid sequence derived from a prokaryotic rRNA gene; (e5) an artificial nucleic acid sequence consisting of a non-naturally occurring nucleic acid sequence; and (e6) a 3′ flanking sequence comprising a nucleic acid sequence derived from a prokaryotic rRNA gene.

The additional partial nucleic acid sequence (e) preferably consists of: (e4′) a 5′ flanking sequence comprising the nucleic acid sequence of SEQ ID NO: 6; (e5′) an artificial nucleic acid sequence of SEQ ID NO: 56 or 57; and (e6′) a 3′ flanking sequence comprising the nucleic acid sequence of SEQ ID NO: 7.

The nucleic acid more preferably consists of a nucleic acid sequence selected from the group consisting of SEQ ID NOs: 58 to 69, and/or a complementary sequence thereof.

According to one embodiment, the present invention provides an expression vector comprising the nucleic acid.

According to one embodiment, the present invention provides a transformed cell comprising the expression vector.

According to one embodiment, the present invention provides a probe comprising a nucleic acid sequence or a complementary sequence thereof, wherein the nucleic acid sequence is at least 90% identical to a nucleic acid sequence comprising at least 15 continuous nucleotides in an artificial nucleic acid sequence selected from the group consisting of SEQ ID NOs: 8 to 57.

### Advantageous Effects of Invention

The nucleic acids of the present invention can be amplified in the same manner as eukaryotic rRNA-related genes using known universal primers for amplifying eukaryotic rRNA-related genes, while possessing nucleic acid sequences that do not exist naturally. Therefore, the nucleic acid according to the present invention enables strict accuracy control of metagenomic analysis based on rRNA-related genes, which is currently commonly employed in the analysis of various microbiota samples containing eukaryotic microorganisms.

### BRIEF DESCRIPTION OF DRAWINGS

FIG. 1 is a schematic diagram showing an illustrative configuration of the nucleic acid of the present invention.

FIG. 2 is a plot showing the quantitative properties of nucleic acids 1 to 12 as internal standards, evaluated using a universal primer set for the ITS1 region.

FIG. 3 is a plot showing the quantitative properties of nucleic acids 1 to 12 as internal standards, evaluated using a universal primer set for the 25-28S rRNA D1-D2 region.

FIG. 4 is a plot showing the quantitative properties of nucleic acids 1 to 12 as internal standards, evaluated using a universal primer set for the 16S rRNA V4 region.

FIG. 5 is a plot showing a correlation between the amount of soil added to the sample and the number of reads derived from nucleic acid 1 to 12.

FIG. 6 is a plot showing a correlation between the amount of soil added to the sample and the total amount of fungi estimated based on the number of reads derived from nucleic acid 1 to 12.

FIG. 7 is a plot showing the copy numbers (actual measured values and estimated value based on the measurements derived from internal standard nucleic acids 3 to 10) of the ITS1 region in a fungal/bacterial DNA mixed sample.

FIG. 8 is a plot showing the fungal/bacterial DNA mixing ratio (actual measured values and estimated value based on the measurements derived from internal standard nucleic acids 3 to 10).

FIG. 9 is a plot showing the number of reads derived from nucleic acid 4 added at various copy numbers to DNA extracted from soil.

FIG. 10 is a graph showing the abundance of microorganisms for each phylogenetic classification estimated based on the number of reads derived from nucleic acid 4.

### DESCRIPTION OF EMBODIMENTS

Hereinafter, the present invention will be described in detail, but the present invention is not limited to the embodiments described in this description.

According to a first embodiment, the present invention is a nucleic acid comprising at least one partial nucleic acid sequence and/or a complementary sequence thereof, the partial nucleic acid sequence consisting of: (1) a 5′ flanking sequence comprising a nucleic acid sequence derived from a eukaryotic rRNA-related gene; (2) an artificial nucleic acid sequence consisting of a non-naturally occurring nucleic acid sequence; and (3) a 3′ flanking sequence comprising a nucleic acid sequence derived from a eukaryotic rRNA-related gene, wherein the partial nucleic acid sequence is selected from the group consisting of partial nucleic acid sequences (a) to (d) below: a partial nucleic acid sequence (a) consisting of: (a1) a 5′ flanking sequence comprising at least 20 continuous nucleotides in the nucleic acid sequence of SEQ ID NO: 1; (a2) an artificial nucleic acid sequence consisting of any one of the nucleic acid sequences of SEQ ID NOs: 8 to 19; and (a3) a 3′ flanking sequence comprising at least 20 continuous nucleotides in the nucleic acid sequence of SEQ ID NO: 2; a partial nucleic acid sequence (b) consisting of: (b1) a 5′ flanking sequence comprising at least 20 continuous nucleotides in the nucleic acid sequence of SEQ ID NO: 2; (b2) an artificial nucleic acid sequence selected from the group consisting of SEQ ID NOs: 20 to 31; and (b3) a 3′ flanking sequence comprising at least 20 continuous nucleotides in the nucleic acid sequence of SEQ ID NO: 3; a partial nucleic acid sequence (c) consisting of: (c1) a 5′ flanking sequence comprising at least 20 continuous nucleotides in the nucleic acid sequence of SEQ ID NO: 3; (c2) an artificial nucleic acid sequence selected from the group consisting of SEQ ID NO: 32 to 43; and (c3) a 3′ flanking sequence comprising at least 20 continuous nucleotides in the nucleic acid sequence of SEQ ID NO: 4; and a partial nucleic acid sequence (d) consisting of: (d1) a 5′ flanking sequence comprising at least 20 continuous nucleotides in the nucleic acid sequence of SEQ ID NO: 4; (d2) an artificial nucleic acid sequence selected from the group consisting of SEQ ID NOs: 44 to 55; and (d3) a 3′ flanking sequence comprising at least 20 continuous nucleotides in the nucleic acid sequence of SEQ ID NO: 5.

In the present embodiment, “eukaryotic rRNA-related genes” refers to genes encoding the 18S, 5.8S, and 25-28S rRNA subunits that constitute eukaryotic ribosomes and the ITS (Internal Transcribed Spacer) region present between the genes. ITS1 region exists between the 18S rRNA gene and the 5.8S rRNA gene, and ITS2 region exists between the 5.8S rRNA gene and 25-28S rRNA gene, both of which are included in eukaryotic rRNA-related genes in the present embodiment.

The 5′ flanking sequence and the 3′ flanking sequence in the present embodiment are selected from sequences comprising at least 20 continuous nucleotides in the following conserved sequences 1 to 5, which are highly conserved in eukaryotic rRNA-related genes (hereinafter, referred to collectively as “sequences derived from conserved sequences”). The conserved sequences 1 to 5 are respectively sequences upstream of the V9 region of the 18S rRNA gene, downstream of the V9 region of the 18S rRNA gene/upstream of the ITS1 region, the 5.8S IRNA gene, downstream of the ITS2 region/upstream of the D1-D2 region of the 25-28S rRNA gene, and downstream of the D1-D2 region of the 25-28S rRNA gene.

Conserved sequence 1
(SEQ ID NO: 1)
TGATTACGTCCCTGCCCTTTGTACACACCGCCCGTCGCTA
Conserved sequence 2
(SEQ ID NO: 2)
AAACTTGGTCATTTAGAGGAASTAAAAGTCGTAACAAGGTTTCCGTAGG
TGAACCTGCGGAAGGATCA
Conserved sequence 3
(SEQ ID NO: 3)
ACTTTCAACAACGGATCTCTTGGYTYYCRCATCGATGAAGAACGCAGCG
AAATGCGATAMGTAATGTGAATTGCAGAATTCMGTGAATCATCGAATCT
TTGAACGCAMMTTGCGCCCYTTGGTATTCCGAAGGGCATGCCTGTTTGR
G
Conserved sequence 4
(SEQ ID NO: 4)
ACCCGCTGAACTTAAGCATATCAATAAGCGGAGGAAAAGAAACYAAC
Conserved sequence 5
(SEQ ID NO: 5)
CCCGTCTTGAAACACGGACCAAGGAGTCTAAC

The sequences comprising at least 20 continuous nucleotides in the above conserved sequences, which are used as the 5′ flanking sequence and the 3′ flanking sequence in the present embodiment, may be selected from any positions of the conserved sequences, as long as they can be recognized by known universal primers for amplifying eukaryotic rRNA-related genes (for example, see Stefanos Banos, et al., 2018, BMC Microbiology, Vol. 18, Article number: 190). The sequences derived from conserved sequences, used as the 5′ flanking sequence and the 3′ flanking sequence in the present embodiment, preferably comprise at least 30 continuous nucleotides in the conserved sequences, and more preferably comprise the full-length thereof.

In the present embodiment, the sequence derived from conserved sequence 1 and the sequence derived from conserved sequence 2, the sequence derived from conserved sequence 2 and the sequence derived from conserved sequence 3, the sequence derived from conserved sequence 3 and the sequence derived from conserved sequence 4, or the sequence derived from conserved sequence 4 and the sequence derived from conserved sequence 5 are used in combination as the 5′ flanking sequence and the 3′ flanking sequence in the partial nucleic acid sequence, and an artificial nucleic acid sequence consisting of a non-naturally occurring nucleic acid sequence is comprised between the combined sequences. In other words, the partial nucleic acid sequence in the present embodiment is a sequence in which the region in eukaryotic rRNA-related gene, between the sequence derived from conserved sequence 1 and the sequence derived from conserved sequence 2 (i.e., the 18S V9 region), between the sequence derived from conserved sequence 2 and the sequence derived from conserved sequence 3 (i.e., the ITS1 region), between the sequence derived from conserved sequence 3 and the sequence derived from conserved sequence 4 (i.e., the ITS2 region), or between the sequence derived from conserved sequence 4 and the sequence derived from conserved sequence 5 (i.e., 25-28S D1-D2 region), is replaced with a non-naturally occurring nucleic acid sequence.

The partial nucleic acid sequence (a), comprising a sequence (a1) derived from conserved sequence 1 as the 5′ flanking sequence and a sequence (a3) derived from conserved sequence 2 as the 3′ flanking sequence, comprise an artificial nucleic acid sequence (a2) consisting of the nucleic acid sequence of any one of SEQ ID NOs: 8 to 19:

(SEQ ID NO: 8)
ATTGTCAGTCTAGCGAATCATTATACCGAAGAACATCCGTTTATGAGAA
CGTGCTACCAATTAACTGTACTAAGCTGTCC;
(SEQ ID NO: 9)
TTACTGATCGAACGTCGTATAATGCTGAGGCATCTGTTATTAACCGTAC
CTTTCAAGGATTACCATGTGGCAACATAAGT;
(SEQ ID NO: 10)
TTGGCCTTCAGTCGAGAACTTGTTGAAACTGTCCTGACGCACTGGAACG
AGCTTCCATTGATTCGCTAGAAATGCCGACC;
(SEQ ID NO: 11)
CCTAGAAAGCTCGCCATTAGCCGCAGTAGTGATTGGACATCAGAGTTTC
GCTCACAACGTCACCGCTCGTTATGGAACTT;
(SEQ ID NO: 12)
TCAGGAAGTGTGTCCCATTGCCGGAGGAGTCCTATTGAATCACGGATTA
CGTCTGTAACGCTGGACCGAGGTTGTATCAT;
(SEQ ID NO: 13)
TCCCGCAAATACCTTTGGAGTGCGTCACTATCTAGGAGTGTGCCGATGA
CTCGTAATCTCCATCCTCGAAGTTGCACGAT;
(SEQ ID NO: 14)
GACACCCTGTTCAGATTAGCGAGCCTCAGTTACACCAGATTCCGAGTTC
GTAAGATCGAGAGGAGCCATCATGGACGTTT;
(SEQ ID NO: 15)
CATGACTGGAAACCCTCTGACGTGTAACTCTGGAAGCTCAGTTATCGGA
AACGGCGCTAAGCTACGTGATCGTAAGCAGT;
(SEQ ID NO: 16)
GCACCTAGCCTTTAACGAGAAGAATGTAGCCCTACGCCATCGGCATGTG
ATTCCATACGATGTTACGAAACCTGAGGCAG;
(SEQ ID NO: 17)
TGCGGAGCATCCTAGTACAATATCCGGTTGCCTATAAGCCCGGTATGCG
CGAATTAACCTAACTGCCAGAGATGAGTTCC;
(SEQ ID NO: 18)
ACGGCACTGATGTTCACCCGCCGTCGATCATACACGCAGGGCGATGACT
CTATGCGAGGCTCCGACCAGTAACAGGCGCT;
and
(SEQ ID NO: 19)
CGTACCTGTCAGCACGCTGTTGACCTTAGCCCGTGGCAACGACTGTGAA
GCCTCCGACACGTACTGAGGGCGATTCCCAG.

Partial nucleic acid sequence (b), comprising a sequence (b1) derived from conserved sequence 2 as the 5′ flanking sequence and a sequence (b3) derived from conserved sequence 3 as the 3′ flanking sequence, comprises an artificial nucleic acid sequence (b2) consisting of the nucleic acid sequence of any one of SEQ ID NOs: 20 to 31:

(SEQ ID NO: 20)
TCATAAGCAGAGCCTTTATCCCATATAAGCTATTGTCACGAAGTGTCACTGTGAACGAAT
GTTCTCTAAACTTACTACGGCTTCAGATGTAACGGATTCAGACTACTCTATTCATAACGGA
CTACAGATTGCGTCAACTACGATATTCTCTTGAGATCACGATTAGCAAGTACCTTTGCAGC
TTGAAATTAACCAGACCTTTCCTTGGAATGCCTATACAGAGATTTATCATACCAGGAGTTC
TCCAGATTACCTAGATGTCTTAACGAGATACAGGACTTACACGATGACTTAGTGTGTTGTT
TGCATCAACCTAACAGTAACTGAGCGAATTGTACCAACGTATTCTTTACCGGAAGT;
(SEQ ID NO: 21)
CATCCTTGGTCTAAGAAAGTGCATGATTTGAGCATACCAATCGCCATTACGATAAAGATC
CTTTGAGTCTAACGTACACTGTGTCATCTGTAAGATACCATTGTCACTACTTCAGTCAGA;
(SEQ ID NO: 22)
CACAGTGTGGATCTGACGAATTACCAAGGCACTCCATGTGTGCCATCTACGTCTCAGGAA
TTGTACCTGCTACCACTAGGCATCGAGAACGCTGCATGTATTCACCGAGTAAGGTCTTCC
AGACTCCGATACCGTATGTGTTCCCAGGAGAAATGTCGCTTAGCCGGTTCAAGCCATCAT
GTGCTAGACTAGACACGTCTATCGCGGTTTACACGACCATCAGTTGAGCCAATGCTATCC
TTGCGGGTCAAACAGAGCTTACGGATCACCCATAGTTGTCACGCCACGTTAAAGTTCCGA
GCGAAACGCTATCTCTTCGAGAGCTGTCCCAATGAAACTCTGCACGGACTTGTATTGCAC;
(SEQ ID NO: 23)
AAGCGTTGGTTCGTTACGCAAGGCTCTACGAAAGCAGTGTCTACTTAGCGTTCAGTGCAG
CGATCCACAATCTCATGGGTATGTCATCGACCAGCTACGACGCAAGTTTCCCAGATCAAG
ATTAGGTGCCCTTCAAGCACGGTTGGAACTCTACCGACAATTACGAGGTCCCAATTACGG
GTGGCAACTATGCTGTACCAGTAAGATCCTGCCGATTCGACGCACAGTCATAACTCAGTG
TACGTGTATCCTGGCAAGGAGGAAGCTCCCTTTACATGCTAGTGCAATGTCCGCAGTTTG
CGAGAGGACTATATCCAGTCTACCACAGGTCAGAGGTTACACCCTGGCTATCTAGTATGG;
(SEQ ID NO: 24)
GCTTCGATTACGATGCCCAAATACGATCCGCGTAGTTTCCACGAGGTCTACAGTACCCTA
TTGTTCGAGGCAGTAACCTGAACCGCGTCTGTCAACAGTTATGTGACGGCAAGTIGTCCA
AGTCCGAGCCATACTATCAGTCGTCTTAGCTCATGGGAAGCTCGCAGTGTTAAGCTCAGT
AGGCAAATTCCAGCGTGATGCCGATCCAGTGTACGAGAATCCTTACATGCAAGTGTCGCA
GGCCAGATCAGTTTCGAGAAAGAGTACGTTCTATCCCTGGCGTCCTCAGTGACTCAAGAT
GAGATTACATCCACACGGTCTCGGTCCATTCGCAAAGTACAGTGTTTCCTTAGCAGCAGG;
(SEQ ID NO: 25)
ATAATCCAGGGTCCACGAGTGAATGCCCTGCAAATGTACCAAGTTCCTGACCTTCTGGCA
TGTGAAGCCGATCTTATCGCTGAAGAGTCTCGAAGTCGCTGACATACACCCGTATTGTCG
ATCTGTTGGCGTAACGGACATACGATGCACTGACAGCAGTTGCTTAGAGCCTAGACACGA
CATTGCCTTGAACGACCTTGCTACTCATAGGGATACCCGACGTAGACGTTTAGTCCTGCA
AGTCGAAAGCCCTTTGTGAGAGTCGCCTTATAGTACCGGATAGTCTCCCAGCCATATTGG
AGAGTCCATATAGCCACGGTAGAATGCTCCGAGGTAACCTGAGTCAAATTGCCGCACTAG;
(SEQ ID NO: 26)
CTGACGGACCAATCTGTATGTAAAGCGGCTATTCAGGAGCCTATCCGACGAGTTGATGCT
TACAAGGCGATCTATCCCTGACCAGTGCTAACCATGTGCATAAGAGCAGTCTCACTCACG
AGTCTCGGTTCCTTAGACGATTCAATGCCAAGTIGTGCCGGAGAACACCTGTTGATCCTC
GACAATGATTCAGTCCACCGGGATGTCTGTAGTTCCCAACGCCAATATGTAGAGCTTCGG
TCCACGAAAGTACCGTGGTAGCCATGATATGACTTACGCCCGACAAAGTTCGGGAGTTTC
TCGCATGTGAAGTTTCCGCAACCATGAGCAAGGTCGTTTGACCTGGAAGTGTATGATCCG;
(SEQ ID NO: 27)
CTCTGATGGACCTGGTGATACACGGTACTATTTGGCATGGTCACATCGGGCATCTGTAAG
ACCTCCAGTTGTAGTGTGCAGAGTTCCCAGACAGTCTAAGACGGCATTGACTATGGCCTT
GTGGTTCGAGAACCGAACATCCAAGAGTTTCGCTCGTTCATGGCGATAACCCTTCAACGT
GTGGTAACCTGTAACGCAGTCAGCTTTAGCGCGTGAATACCTTGAGGCAATACACCGAGT
TGTGCTACCCTAGTGATGACAGAATGGCACCTTATGCTCCGGTACACCTACGGAATCATG
CAAGTGGAATCCCTTTCGAGAGCAGGCTCAGTTTAGTTGCGAAGTGATCTCCGCATTTCC;
(SEQ ID NO: 28)
CTTCTGAAACTATGACGCGCCAACCGGAATCGTGTAATGGATTGACCTACTTGCTCGGAC
GACGGATAACGCTGTATGCAAATGTGCCTGTAACTCGGCTCTGCGAACTGCTCTGATCTA;
(SEQ ID NO: 29)
TAGGTCACGCTAGTACCAAGGAGACTCAGACCTTACAGCTTGCTTGCAGACAGATCGGAA
TCCCACAGCAGAGTTTAGACGTTTGGAGACAGTCCCACTTCAGTCGTTGGATGCACTTAG;
(SEQ ID NO: 30)
CCTGGCGAATGTCTAAGGCGTCCATATCCGAGGTGCAGCGCGTTGCCTGACCATTAGGCC
CGTATAGTTCGGCGTGACCGAGATGCCGCTCAGTACGACGGTCTAACAAGCTGGCCGCAC
TTGCCAACCTGTCGCGGACTGTCTTAACGGTGGCCCGACTTGCTACCACACCCGTGGGAT
TGTGCTACGAAGCGTCCCGAAGGTCCTCAGCCCAAGAGTCCTGTAGTGAGTACCCGGAGC
CTCGACCCTGATGTGATCCGACCAGATTGGAGCCGGTGACCCTCAGACGGAGTCAAGGTC
CTACCTGTGAAGCCCTGACGGCGTGGATTCCTGCTAGAGCCAAGGAGAGTGTCCCGCTAC;
and
(SEQ ID NO: 31)
CCATACTGCGAATGGGAGCCGCCGGAGGTAAGTCCTTTCCCTGATGACCTTGCGCGTAGG
GCCGGGTAAGAGCTTCTCCACTGACTGTCAACCGTGGGCACGCCGAGGATGCTACTCATG.

Partial nucleic acid sequence (c), comprising a sequence (c1) derived from conserved sequence 3 as the 5′ flanking sequence and a sequence (c3) derived from conserved sequence 4 as the 3′ flanking sequence, comprises an artificial nucleic acid sequence (c2) consisting of the nucleic acid sequence of any one of SEQ ID NO: 32 to 43:

(SEQ ID NO: 32)
AGTTGTCTGCCAGAAATCATTGAACATTCCGACGAATATCGACATGGTTGCTTATCTAAG
ACCTTAAACGGTACTTGGTTAGCTGATCGCAATACTTGAAAGACTTGATCCTGTACTTACC
TGGACACGATGTAATAATCTCACACAGTTATGAGAAGCTGGTTGCACCTAAATAGTCAAT
TAGCACGTAGTAACGTAGACTTGCCACTGATGAAACATA;
(SEQ ID NO: 33)
CATTGAACACTTCGTAAGGTACACCTATGGATCAACGATTAAGTCTCGATACCGTAAGAT
GGTAACTCTAGTCAGTGATAATCAACAGCGTAGTACATTCGTAAGCAGTCTTGGACATTA
CTTTCTGAGTGCAACATTCAACGTCTAAACGGGTTAAATCTCTCATAACGGAACTTGTGTG
CAACAGATGCTATATGGTATGCAAATGCGATACACTTTG;
(SEQ ID NO: 34)
ACTATGAGGCCCACAGTTACGAACGACTAGACCACTGTCTTACGAGTGTCGCACCATAAG
ATGGCGAGTAATCCGCTCAATCCACTGGTTCCTGAGAAAGAGCCGGAAATCTGAGGTCAT
TCTGCCCATGATAGCTGGAAACACCCGAGTCTCTAAGTGTGAGTAGCCTGATCTACTGCA
AACGCCCGATACATATCGTGAGAGTCTGCTAGGACTGATC;
(SEQ ID NO: 35)
ACCGTAAAGCTAGGTCAGGTCTTCACTGGGCAACGACATAATGGGTAACTCACTTCCAGC
CTACATCAGCGGTGTCAAAGGTAGATGCCTATCGTACCACCCACAATGCTCTAGGGTTTC
AGAGAAGCTGTGTCTTCCGATGGTCACCAGATGGATTCGACTCAAGGTCATACAGGAGTG
TCGCGTAACATAGCCTATGCAACCGTTCGGTTAAGGACGT;
(SEQ ID NO: 36)
AACATGCTGCGTAGTACGTCGATCACCAAGCTATGAGCGTTGTCAAAGGAGTGTCAACCG
ACGAGTCCAGGTTTCATCACCTTGCTAGGTATCCACAGGTGCATTAGGCGGCTAAGTCTT
CCACATCGTATTGCCGAAGTGTATCGCCCAGACATTCAAGCTGTCAGAACTCTGCGTTAC
AGAACGTGCCGTCAAGATTCAGGCTATCATCCGTGAACCA;
(SEQ ID NO: 37)
AGTGACAGTTCACGGTAGCAGCTAAATCTTCGGGCATCACGAGTACATGAGTCTCCCATC
GTTAATCCAGCAAGCCGATGTGGAGCTATTTCAACGGGACGTATATGTCGTCCATCCGAG
TTGCGGACTATCTACAGGGTGAATTATGCGACTGACTGCCTTGCCACTACGAAACAGTGC
GTTCAAATTGCGCTAAGGGCGTGCGAATACTTATGCAGGC;
(SEQ ID NO: 38)
ATCTGACAGCCTTCTACGAGCCTGCTGAATCAGATGAACCACTTGGTCGCAATGATCGCA
AGGTCGGGTATATCTTCACGGTTAGATCCGAACTGCTCCACTGGGTACAACACACTGACT
TGGTAACTCGGTCATACACGTCGGGAACATAACTGCCTGTGATAGCACGCACTCTTAGGA
CAGTCGCATTCTCTAGGTCATGGAATAGCGCAACATCGCT;
(SEQ ID NO: 39)
AACTTAGGGAGTATGCCGTCGAACATCGCTCGTGAGTAACTTATCGTGCGGATACACCTC
GTACATGCCACTCGGTACTTAGAATAGCTGGTAACCTCCGATGCTCGCAATGCGTAGTTC
TGGATTCCAATGGACCAACGGTCATTCCTGGGTGACAAAGCAATCTCCTGTAGCAGGTCA
CAGTTCTCGTCTCGCAGTAACGAAGTCCTCTTACGTCATG;
(SEQ ID NO: 40)
TCCACGTAAATCAGCGCGTTATGGGTCTGACGTAAGCACAAGGGTCCTATACACGCTACT
CTGGTTATCCCTGAGAAGTCGGTTACCATGTCACACAGTCAGGCTATATGCCCTCACGTTG
ATTCGAGCGAAGTTACTGCACCAAGTCTGGCGTAGTTAGTGTTCCGTAGAGCAAGTCACT
CAATCCCGAGCAAAGTGTCGTGATGCTGTTCAGCAAGAC;
(SEQ ID NO: 41)
CAGGGTTCCCTAGTAAGTACGATTCCAATACGCGATCCGAATGCGGCGTTTCCTAAGCAA
GGTATAATCTCCTGACGAGGAGTCGGGTCCATAAGGTTTCCATAGTTCACCGTGAGACTG
CGATGGTCTGCCAATGTTCACTTCAAGTCCGTAAGACACGGCAAGAGCCTAGCATCTGTT
CGTTCAGAGTCATGGTATCGGACAACTGCCTGATCTTCGA;
(SEQ ID NO: 42)
GCGGACGATGCCTTTGTCGATAATGCTCCCGCTGTAGGCCAGCGCCAATCGGCTGTGCAT
TTAGCGAGGTCTCACGCCAGTGCGAGTACGAGCCTTCCTCCTAAGCGTTCGGTCGGACAG
GACATCTGGATCGCGGAACCCTAATCCCGTGGGACACCGTCACTTGGTCGATGCGCGTAG
CTTGTCACCGCAGGGACTGAGAGGTCAACCCATGCGACTG;
and
(SEQ ID NO: 43)
GGCAGCTTTACGGTTCCCAGTGCCTAATGAGGACGCCTGGGCGGAATCGAGCCTTCGGAA
AGACATCTGCAGCACGGTGCCTGCAACCTGTCGGTGACGTATCAGGACCTGGTGTCCACC
CGTTGTCAGGGCTTCCAAGGTCAAGCAAGTGGTGACCGGCCATGCGTGGTCGCTTCACAG
AACATCACGGCAGTCGCCGTATCGGCCCGAGTGAGACTAG.

Partial nucleic acid sequence (d), comprising a sequence (d1) derived from conserved sequence 4 as the 5′ flanking sequence and a sequence (d3) derived from conserved sequence 5 as the 3′ flanking sequence, comprises an artificial nucleic acid sequence (d2) consisting of the nucleic acid sequence of any one of SEQ ID NOs: 44 to 55:

(SEQ ID NO: 44)
GAACGATTGAAGATGTACTCAGATATTCATTGATGGGCCTACGTCTACTTACTATGGGAA
TGTAAATACTCTGTTCCAGCCTAAGGTTAGCTTTGCGAATACAAATGTTCTTATCGACGCA
CAGTCATACGGATTACGATCAAGTTAATGGTTACTCCCTACCGATTATTGCATCCAGATCA
TATTGAGAGGAATCACCTGTACGGTTTAGAAATCAGCTCTACTAGAAGACACTATTGCCA
TACGTCAAATTGCAGTGAGTTTCACCAAATCATGGAGATGTTACCCAGTTAGCATACAAC
TCTTTGCACAAGTGCATAATGTAGTCCCTATGTCACAAGGTTATACGAAGCATGTCAAAT
CATCGCCTTTAGTTACGATGTAGTTCCACAAGCGAAATTAGTTTCCGAAATGGTCAAGCA
TCCAAGTTTAGCTCGAATCTTTAAGGAGATACTCGAAGTGCCTATATTACGGAGGTATTA
TCATGTAGCAAGCGTTACCTAGCTTATTAGTCCACGAATCATGTGTTAGAAGTCGTCAAG
TTCATGTTATCCTACCAG;
(SEQ ID NO: 45)
GTAAAGCTATTAACCGGAGTGAATCCTTCATTAAAGTCGCACAAGCTGTATTACCGTTAC
GCAACGTATTTGATTGACCATGTGAACAGAAGTACCCTATTGACCTAGATTATGCAGCAA
TGCCTAAGACTATTTGCCTAATTCGGGCTATTTAGACCAATCCTCCATGATGTATATCAGT
CAAGGCTAGTTTGGAACATACACGAAAGTCCTTATGTAGTAGAGTGCAATTCTCGTATCC
TTCAACAGTGTTATCGAGTATCGAACGATTATCCTATGGGTATCCACTTATAGAACGTGTG
TAGACTAACCTGTAAACGATGTCTCTGAAAGCAAGACTACTTATCTGAGATCGGATGTTT
AAGACGCTATGACACCATTAACTTATGCCAGTGCTAGTCATTATGACCACGATTTGGAAT
TTATGGCTATCGCCACTATGAAATGCTAAGCTACCTGAACAATTTGTACGCAGTGACAGT
AGATCCTTTGATCCAGAACTTATTAAGAGCTGACCCTATGAAACGTGATGTCCTATTCATT
ATTACGGGAAACCGTAG;
(SEQ ID NO: 46)
TCAGGCTATATTGAGGCACCGCCTGGCTAGTAGATTACGACAGCTATAACTTCGGGCAAG
CCGGTTGATCCAACTATCGAAACCTCGTTAGAGCAGTGTGTGGCCTAATGGCATACTGGA
ACCTATCTGTTACGCCGAGAACTCGTGAGCAACTCAGTCTCATAAAGTCATGGTCCGCAC
TGATGCTGCACAAAGCTACCGATTGATACGTTCGCCGACTGTGATGCGTGAATCATTCCG
TCAAAGTGTCCACCCGTGTAGGCATTGGTATATCGACCGATCCAAGAAGCGACGCTTAGT
ACGCGATTACATTGGGCAGATGGTACAGCTCCCATAAACGCTAGGAACTGTTCGCAAGAG
TCCTGTGTCAGAGTCAAGGATACCGTTCAGAGGCAAACTGACCGTCATTCGTGCTAAACG
ATGTGATCCGCCCTTTCAGACGCTAGTGTTACCTGGAAGAAGATTGGCGCTACCTATGTC
CCATACAGCGACAAGGTCTTGTAGAAGGCATGTCAAGCTCCCTAAATGGCTCCGCTAAAG
TACGTGTTGAGGGTCTCCAA;
(SEQ ID NO: 47)
GCTGCTTAGCCTATACCGTAATCGGTGTGCGTGAACACTAGCCAGGTACTGAATCTAGGA
TCGCTGTGGATCTAACCAGTCCGCTACGACAAGAGTTTACTAGGACCGCCTAAATCATCG
GCGCTTACCGTTAAGAAACCTGTCCGGCGACATATACAGTGCCATTGCGCTTGAGAATCA
TGCTGTGCGAGAGACATACACGGTTCCGAGTTGACATCTACGTGAAGGGCATCTTTCGAT
GCTGACCCGAAGTTTATCTGGGAAGCTACGTCATTTGCCTACCGCTGCGACTAATCTTTGC
AGACGACATGCTATGAGCTTGCTGGACCACGAATCGTTACCAGTCATCTGAGACACTTGG
CATACGCTTGGGCTTGATACACCTATGGATGGGATACACTGATCGGCTGCCGCATAATTT
GCTACGCCTTACAGAGAAGTGCAGTCTACCGGCTGTTAATACTCCGGCTTTACACGAGAA
GCTACTGAGGGCCATTTGACACAATCGCGTGAGTTTGCTGATCTGACATGGGCTGAAACA
TGAGCCTCCGAACTATCGT;
(SEQ ID NO: 48)
TACGTGAGATCGGTCCGATATGAGCTGTCCACAATAGCCATAGACTAGGAGTCACCCTTC
GAGTGGTTCTAGCACATCCAGATGACACACTAAGTGCCCTGTTCGGGACTTGTAAAGCAC
GATTCCTTGGTTAAGACGCCTCCCAGTCAGTATCATGGTCGTAAAGTTCGTCCAGTGGTCA
ACGCTCTTCGTCAAGCGATAAGTTAAAGCCGGTAGCTGCTCAAGCCTGCCATACGGATTA
GTTCAAACGAGCCTGTCGTGTACGTTCTCCGCACAATGTCTAACAATGGTACGGTGCAGA
TAGCTTCCGCCCAGGTTATTAAGGCAAATTGGCCCATCCATTCTGTCGGTCGGCAAACAG
TTCCTGAAATTCCGCTGAGGTTGTAAGACCCGGTCTGAATAGCCAGATCAATACGTCGGT
GCTGATGAGTGCCATCACAGTTTCTCTAGGATAGCGCACGTTCATGTCGCGTAACGCATC
TAGCATTTAGGTGCAACGGTACTACGTCCACCAGTAGGAAGTTCGCATAAACGGTCACCT
TAGCCTGAGTAGCCGTCAA;
(SEQ ID NO: 49)
ATGTCCAACCGAAACTCGTGATCTTAGTGACCGCACGGATCTGTCATTCGAGAAGCGTAG
AGACTTATGCCTGGGCCTTAACTTGTGCTCAGTAGCCTCAAGAGAACTGCCTCCTGTCTAT
TACGGGTAAACTCCTGGTGATCCAGAGACGTAGTGTCAGAACAGCCTAGATGTGTTGCCA
CGACCTGTAAACGGCTTTCTTACGACGCAATGCTGATGGTGACTGGCGATTAACGAACCG
AATCATCCTGTGTGCATCCTACGGTGTGCCATTTGAACCAGAGAGTATCTTCGACCACGA
TCTGCAAGGGTGTCATGCTTGACCTAGAGTACCACGTTCAGTTGCCTCATAGGGCTTAGC
AGCGTATTCATGCGACTTGCGATAACGATGTCCTGTACGGACGTTCCATAGTCCGACAAA
CCCATGTATGTCTGCGAGAGGTTAGCCAAGAGTGCTTACTCCACCTAGTGAGATGTAGCG
ACAACGACTGTGAGTGTACGACTCCTTAGGGTATAGCGTTGCCAAACTTCCCAAGGTAGG
GAGCCTTTCCCATTACGAA;
(SEQ ID NO: 50)
TCCACAGTATCATCCGATGGAGCGATTCGCATACGACAGTCAATGGCTATTGGTCAGGAC
CTAGCTTCCAAGTCAAGGGAAGGTTTCAGGATCGTCGCATCGTACTTTCCTACGAAGTGC
CTAAAGGGATCACTCTCCGAACGGTTTGTATCAGCGTGCAGATGTACCTGTTACGCCAGA
GGAATGACATTCTACCCGAGGGATCTTACAGTCCGGGATTTGTGCAATCACAGTTGGGCT
CTAACGTCAAGCGAGGTGTATGTCCCATGAATAAGGACGGCTTTCTCAGGCCAAGAAGTC
TACGCAGAAGTTACCCAGCTCGTTTACGGTGTCCACTCAAAGTCTAGCATGTTCCGGTGA
CCTAGTTGATGGCAGTAGCAGTACCATGACAAGAGGCTTCCGATTATCCAGACCCAGTTG
TGGGCTAATATGAGCAGCACCCTAGTATTTCGCGCAATGCCGGTTATATGAAGGCCACGT
ACAAGTTTCTCCGCGCATGTGTCAGATAGTATCCGGTTCCACAGCATAAGTCCGCCAGTT
GGTTCACTAAGTTGCCGACA;
(SEQ ID NO: 51)
TATTGACGACCGTTGCCAGAGAGCCATCACTTGGTTTCGACTATAACGACAGATCCGTGG
CCTCCTAAAGTTGCGTATGCAGTATCGAGATGTACCCTGCGAACCGAGTGTACTAACGTG
TCTGAGGAATCCATTCCCGTATCGGGCACAACAGTATGTGTCTTCCAGATAGAGGGCCTT
TGCTGACGAAGTCCTAGACTATCGCTTAGAGACGCCTACAGACCAGTAATCGTGACCTTC
TACCTGAGATGCCGTGAACATAGGTGCTAATCCGAGAGCATGTGTACGAACTCCGAACCT
TGCCATTAAGGGATGAGCCTACTGAACTACCGCTGATCGTGCGAGTATATCCTGCTGCTA
ACGTAAACTCCTGAGGGCTACAGCTAAACAGCTTGGACCTAGTGTCATATCGCCGTTCCA
ACTGACTCCTTGAGAGACTGCGTAAGATTTCCGCCGACATTGCCAAACGCTAATTGCCGA
TGGTGTAAACGACCCGCATTCCATTGGTTGCTAAAGCCTCGTAAGAATCCGGGCTGACTA
TCATGTGAGCTTGACGCTAC;
(SEQ ID NO: 52)
AGGTCCTCAGAGGCTAATGTTTCATGCAATGAGATCCCGCGTGGACACCACCAAGATTCT
ACTGTTGTCAAGATACGGGCGACTCGACATGGAGCTACTATTCTATCAGAAGAGCCCTGC
CAGGCGTTCAATCGCATTTCCATTTAATGGCTGACTCGCGCAGACGAAGTCTCCTAGAGT
TAAGTCTTACGAGCACCGCTTGTGTGAGCACGATCATACGATACTGACTAAGGCGTCACC
GAGTTTCAGACCCTACGACATGACTGTCTTTAGGCCAGAGTCTACTAGACCGAGCTTTGG
ATGCCAACCTTTCCGAAGTGAGATTTACCCACAGCGTTCGTGTGTTCGACTAACCCGCAA
AGTGTTACCATAGGCTGGTCCTATTTCGCAGTGGCTAGAGAGCAATGTTCCAGGATGTGC
TACTACTTGCCGTGAGCTAGACATACCGATGGCTAAGTGGATACGTTACAGGCGCACGTA
GTTCTAACCGGCTTATACGGATAACCTGACCCGAGCGTTATTCTTATGCCGCAGAGAGGT
TTCTTACCCGAAGGCACTAG;
(SEQ ID NO: 53)
GTCACATGCAAGCTGTTTCCTTCTACATGACGAGCCTCTGCGATAGGTGAGTATCCCACTC
ATTGATAGCTGCCGCAAGTCAGGAGAATACGTCCGTTAGTAAACTGTCCCATGCCGAAGC
TCAAGACCTGGAAGTCCTTGATAACTGGCACACTCTGAGCCAACTGAACGTGTACGCATT
ACAACTCCGGTGTTAGCCTGCTTAGCTGAACCAGCAGTAATTGTTAGGCGTCCCAACGAT
CCATGATCCGCGTGAAGAAATCTTTAGCGCCCATAGGCAGTAAGGTAGCCCGACATAGTG
TCTATTAGGCCCGAAATCCCTTAGGGAGCCCAATACATGATCTTAGCCGAGTCGTAGGAA
CGTCCATCTCGAAAGTCGTTTGCTAGGGCAATCCAAGTCTCGATCCCGATAAGTTCTGGCT
AGGTTGACAAAGCGTCCAGATCCGACGAGTAAATGGTCCCTGTTAATCCGATAGTCGCGC
ACCACGGTGAATATAGTCCGATGACATTGACCTGTACCAGACCGCGTCTCAAATTGACGA
AAGCGATGTTCGTAACCG;
(SEQ ID NO: 54)
GGTGGAAAGCTCGTCTCCCAATGCCATTAGCCTCGGCGGAGCGATAGCAGCTCCTCTGGA
AGCATCAGTGCGTCTGCCCAAGGCGTTCCTCGTCGGTACAACGTAGACTGCCGCTACGGA
CGGTGTCACCAGGGATACACTCCATAGCATCCGGGTCGCAAGGTGTGCGTGCCAACTACC
CGACTTCTAACAGGGCTGGCCGATACTGCGGGCTCAAGTGACTCAGATCCTGAAGGGCGC
ACCACGTCGCGGACTACAGTGTTCACATGAAGCGCGGTCGTGCAGCGCATGGTCCATACC
AACTGCCTAGTACGCGGGACTGGCGTCGAATCGACTCGTCCTTCGGAAACATGACGGCGC
GGCCTAAGCGAGAACTCTGCTCGTGTCCATCAACGGCTGGCGGCGATATGTCCTGACCTC
AGCCATAGTGCCTACCTCGGGAGCGTTCAAGCGATCCTCGGTCTTAACGGGCGAACTCGG
GCTCGAAAGCGAATGCCTCCCTAAGCTCTTCGGTGGCGGACGCGGAATCATAGCTCAGCG
AACTCTCACGGTTGCAGGCG;
and
(SEQ ID NO: 55)
GTCGTGACACGCTTCGACGATTGAGTCGCCGCCTACGACTGACGATCTTCCGCCTGTAGC
TGGATGTGCCCGATCCGTGAGGACATTCCCACCTGGACTGACTCGCATGGAGACTGCCAC
GGTGATTCGCAACAGCCCGTAGAGGCTTCGTTCGACCACCCGATGCTGAAAGCTGCTGCG
CTGATCTGAGACCTCGGAGGGCGTAAACTGGACACCTGCCACTCGGACTGTGTTCGCACG
TCGGCTTCATAGCCACTGGCAACCGCGCTTGTGTGCAGACGGAACCCTTTAGTGCCTGGC
GATGACCCTACTCCCGGTGAACGGCAATGCAATGGGCCTGGAACTGTGACGCTCCCGTAC
CTTCCCTTGAGAGGACCTGGCATCTGGACGCAACTCCTGGGTGTGACCTGTGAGCAACGC
CTCCTACTGGGTATAGCCCGCGCTTAGACGCTGCTAGAGCCGGAGACATACGATCCCTGC
GCTTACACGCACGCGATAGGTGCGCTCGATAATCTCGGCCCGGTAGTGCAACCTGACCAG
CGGTAGACCTTGATGACGGC.

The nucleic acid of the present embodiment comprises at least one partial nucleic acid sequence (a), (b), (c), or (d), and/or a complementary sequence thereof. That is, the nucleic acid of the present embodiment may be either single-stranded or double-stranded. Also, the nucleic acid in the present embodiment may be DNA, RNA, modified nucleic acid, or the like, and the nucleic acid in the present embodiment can be prepared using one or two or more of these. Accordingly, the nucleic acid in the present embodiment may be, for example, single-stranded RNA, single-stranded DNA, double-stranded RNA/DNA hybrid, double-stranded DNA, or the like. In the present specification, a nucleic acid sequence composed of DNA is shown, but it can be appropriately read as other nucleic acid sequence, such as RNA, and the nucleic acid in the present embodiment includes these. In that case, thymine (T) and uracil (U) may be appropriately replaced.

The nucleic acid of the present embodiment preferably comprises two or more different partial nucleic acid sequences selected from (a), (b), (c), and (d), and/or a complementary sequence thereof, and more preferably all of the partial nucleic acid sequences (a), (b), (c), and (d), and/or a complementary sequence thereof. The order of the two or more partial nucleic acid sequences arranged is not specifically limited. Here, when the nucleic acid of the present embodiment comprises the partial nucleic acid sequences (a) and (b), (b) and (c), or (c) and (d) continuously, the 3′ flanking sequence of the former and the 5′ flanking sequence of the latter may partially or entirely overlap, but such overlapping sequences are preferably not duplicated in the nucleic acid. In other words, the sequence derived from each conserved sequence is preferably unique in the nucleic acid of the present embodiment.

The nucleic acid of the present embodiment may further comprise an additional partial nucleic acid sequence (e) consisting of: (e4) a 5′ flanking sequence comprising a nucleic acid sequence derived from a prokaryotic rRNA gene; (e5) an artificial nucleic acid sequence consisting of a non-naturally occurring nucleic acid sequence; and (e6) a 3′ flanking sequence comprising a nucleic acid sequence derived from a prokaryotic rRNA gene.

The nucleic acid sequence derived from a prokaryotic rRNA gene used in the nucleic acid of the present embodiment as (e4) and (e6) may be any highly conserved sequence in the prokaryotic rRNA gene, but preferably comprises a sequence that is recognized by universal primers used in metagenomic analysis of prokaryotes. That is, the sequence (e4) in the nucleic acid of the present embodiment preferably comprises at least 20 continuous nucleotides in a sequence upstream of the V4 region of 16S rRNA gene:

CACCGGCTAACTCCGTGCCAGCAGCCGCGGTAATACGGAGGGTGCAAGCGTTAATCGGA ATTACTGGGCGTAAAGCGCACGCAGGCGGTT (SEQ ID NO: 6), and more preferably comprises the full-length thereof. The sequence (e6) in the nucleic acid of the present embodiment preferably comprises at least 20 continuous nucleotides in a sequence downstream of the V4 region of 16S rRNA gene:

GTGGGGAGCAAACAGGATTAGATACCCTGGTAGTCCACGCCGTAAACGAT (SEQ ID NO: 7), and more preferably comprises the full-length thereof.

The artificial nucleic acid sequence (e5) in the nucleic acid of the present embodiment may be any sequence, as long as it is a non-naturally occurring nucleic acid sequence that is a different sequence from the artificial nucleic acid sequences of SEQ ID NOs: 8 to 55, but is preferably:

(SEQ ID NO: 56)
ATAAGAGCTTTGAGCCCACCCGCATACTGATTTGACTGCCTTAACTTGGT
GAAGCCCTCGGACGGAAACTTGACATCTCGTTCTATCTGAATGAGCGCGG
CACAGCTTGAGTCTACTTGGAATTGCATTAGCACCGGCCTGCCTTACAAC
ACTGTTGCGTATTGGACTAACTAGCGGCCT
or
(SEQ ID NO: 57)
GTAGTTAGGCAACTCTAGGCGGCAACTGCTCATCAACTAGGAGTACAGTC
AATCTGACGGACGCGCTACTGCATACTTAGTCATCTACTGGTTCCAGAGC
CACGGGTCATCGTAAATTGGGTATTCCGAAATGGCCCACACGCCGTTCAC
GTTTCAAATGATTGGCATCTAGGGACACCT.

Specific examples of preferable sequences of the nucleic acid of the present embodiment can include the nucleic acid sequences of SEQ ID NOs: 58 to 69. The nucleic acid sequences of SEQ ID NOs: 58, 59, and 62 to 69, comprise all of the partial nucleic acid sequences (a) to (d). The nucleic acid sequences of SEQ ID NOs: 60 and 61 comprises all of the partial nucleic acid sequences (a) to (d) and further comprises additional partial nucleic acid sequence (e).

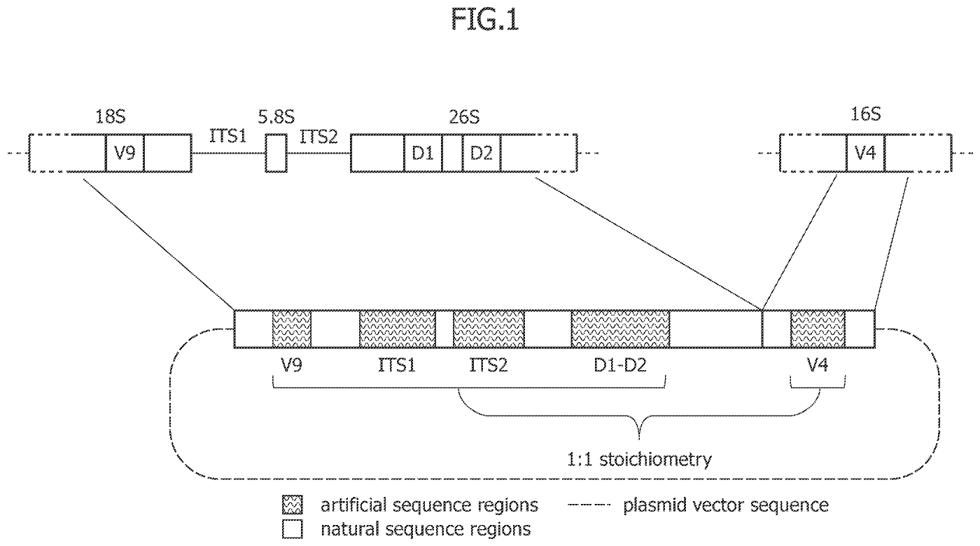

FIG. 1 shows an illustrative structure of the nucleic acid of the present embodiment. The nucleic acid sequence comprising partial nucleic acid sequences (a) to (d) may be a eukaryotic rRNA-related genes sequence in which the 18S V9 region, the ITS1 region, the ITS2 region and the 25-28S D1-D2 region are replaced with non-naturally occurring nucleic acid sequences, and a nucleic acid sequence comprising partial nucleic acid sequence (e) may be a prokaryotic rRNA gene sequence in which the 16S V4 region is replaced with a non-naturally occurring nucleic acid sequence. Also, the partial nucleic acid sequences (a) to (e) each are preferably contained at a ratio of 1:1 in nucleic acid molecules. Also, as will be described below, the nucleic acid of the present embodiment can be incorporated into an expression vector to be introduced into a cell.

The nucleic acid of the present embodiment can be easily prepared by any conventionally known nucleic acid synthesis method.

The nucleic acid of the present embodiment may be added to a sample to be analyzed at an appropriate timing. For example, the nucleic acid of the present embodiment can be added to a microbiota sample before extraction of nucleic acids, and in this case, it is possible to control the accuracy of the entire analysis from nucleic acid extraction to amplification. Also, the nucleic acid of the present embodiment can be added to a nucleic acid solution extracted from the microbiota sample, and in this case, it is possible to control the accuracy of only the amplification reaction of the nucleic acid.

Here, “microbiota” means a collection of multiple microorganisms that exist in a certain environment. The microbiota can be composed of, for example, at least 100, 300, 500, 700, 1,000, or more types of microorganisms. The microorganisms constituting the microbiota may be any class of prokaryotic and/or eukaryotic microorganisms and may include, not only known microorganisms, but also unknown microorganisms. The “eukaryotic microorganisms” mean any unicellular or multicellular eukaryotic organisms of a size that cannot be visually determined, and examples thereof include fungi such as yeast, mushrooms, and mold; microalgae such as Euglena, Scenedesmus, and Volvox; protozoa such as Paramecium caudatum and amoeba, but there is no limitation to these examples.

The present invention according to a second embodiment is an expression vector comprising the nucleic acid as disclosed above. The expression vector that can be used in the present embodiment is not specifically limited, but may be a pUC19 plasmid vector, a pT7Blue plasmid vector, a pGEM plasmid vector, or the like. The expression vector of the present embodiment can be added to a sample to be analyzed like the nucleic acid of the first embodiment. Alternatively, the expression vector of the present embodiment can be used by introducing it into a microorganism cell.

The present invention according to a third embodiment is a transformed cell comprising the expression vector. The cell that can be used in the present embodiment may be any microorganismal cell, e.g., E. coli DH5α, E. coli HB101, E. coli JM109 (NIPPON GENE CO., LTD.), etc. The introduction of the expression vector into a cell can be performed by a well-known method in the art according to the type of the cell, such as chemical transformation or electroporation.

The transformed cell of the present embodiment can be added to a microbiota sample before extraction of nucleic acids, and this enables the accuracy control of the entire analysis from nucleic acid extraction to amplification.

According to the fourth embodiment, the present invention is a probe comprising a nucleic acid sequence or a complementary sequence thereof, wherein the nucleic acid sequence is at least 90% identical to a nucleic acid sequence comprising at least 15 continuous nucleotides in an artificial nucleic acid sequence selected from the group consisting of SEQ ID NOs: 8 to 57.

The probe of the present embodiment may be any oligonucleotide that specifically hybridizes with the amplified product containing the artificial nucleic acid sequence. Accordingly, the probe of the present embodiment comprises a nucleic acid sequence or a complementary sequence thereof, the nucleic acid sequence being at least 90%, and preferably 95% or more, identical to a nucleic acid sequence comprising at least 15, preferably 20 or more continuous nucleotides selected from any position in the artificial nucleic acid sequence.

The probe of the present embodiment is preferably labeled with a labeling substance (e.g., fluorescent dye such as FITC or Cy5) for detection of the corresponding amplified product.

The probe of the present embodiment can be easily prepared by any conventionally known nucleic acid synthesis method and can be further labeled by a conventionally known method, as required.

The probe of the present embodiment can be used in combination with the nucleic acid of the first embodiment, the expression vector of the second embodiment, or the transformed cell of the third embodiment, so as to enable accuracy control of the analysis of microflora samples.

### EXAMPLES

Hereinafter, the present invention will be further described with reference to Examples. However, these Examples do not limit the present invention by any means.

### 1. Design and Synthesis of Artificial Sequences

The nucleic acid sequences shown in SEQ ID NOs: 58 to 66 were designed as below: nucleic acid sequences (nucleic acids 1, 2, 5 to 12 (SEQ ID NOs: 58, 59, and 62 to 69)), in which the 18S V9 region, the ITS1 region, the ITS2 region, and the 25-28S D1-D2 region in the eukaryotic rRNA-related genes are replaced with non-naturally occurring artificial nucleic acid sequences; nucleic acid sequences (nucleic acids 3 and 4 (SEQ ID NOs: 60 and 61)), in which the 18S V9 region, the ITS1 region, the ITS2 region, and the 25-28S D1-D2 region in the eukaryotic rRNA-related genes are replaced with non-naturally occurring artificial nucleic acid sequences, to which a prokaryotic 16S rRNA gene partial sequence with the 16S V4 region replaced with a non-naturally occurring artificial nucleic acid sequence is added; and prokaryotic 16S rRNA gene partial sequences (nucleic acids 13 to 17 (SEQ ID NOs: 70 to 74)), in which the 16S V4 region is replaced with a non-naturally occurring artificial nucleic acid sequences.

Nucleic acid 1
(SEQ ID NO: 58)
TGATTACGTCCCTGCCCTTTGTACACACCGCCCGTCGCTAattgtcagtctagegaatcattataccg
aagaacatccgtttatgagaacgtgctaccaattaactgtactaagctgtccAAACTTGGTCATTTAG
AGGAACTAAAAGTCGTAACAAGGTTTCCGTAGGTGAACCTGCGGAAGGATCATTAtcataagcagagc
ctttatcccatataagctattgtcacgaagtgtcactgtgaacgaatgttctctaaacttactacggc
ttcagatgtaacggattcagactactctattcataacggactacagattgcgtcaactacgatattct
cttgagatcacgattagcaagtacctttgcagcttgaaattaaccagacctttccttggaatgcctat
acagagatttatcataccaggagttctccagattacctagatgtcttaacgagatacaggacttacac
gatgacttagtgtgttgtttgcatcaacctaacagtaactgagcgaattgtaccaacgtattctttac
cggaagtAAACTTTCAACAACGGATCTCTTGGTTCTCGCATCGATGAAGAACGCAGCGAAATGCGATA
CGTAATGTGAATTGCAGAATTCCGTGAATCATCGAATCTTTGAACGCACATTGCGCCCCTTGGTATTC
CAGGGGGCATGCCTGTTTGAGCGTCATTTagttgtctgccagaaatcattgaacattccgacgaatat
cgacatggttgcttatctaagaccttaaacggtacttggttagctgatcgcaatacttgaaagacttg
atcctgtacttacctggacacgatgtaataatctcacacagttatgagaagctggttgcacctaaata
gtcaattagcacgtagtaacgtagacttgccactgatgaaacataGTTTGACCTCAAATCAGGTAGGA
GTACCCGCTGAACTTAAGCATATCAATAAGCGGAGGAAAAGAAACCAACgaacgattgaagatgtact
cagatattcattgatgggcctacgtctacttactatgggaatgtaaatactctgttccagcctaaggt
tagctttgcgaatacaaatgttcttatcgacgcacagtcatacggattacgatcaagttaatggttac
tccctaccgattattgcatccagatcatattgagaggaatcacctgtacggtttagaaatcagctcta
ctagaagacactattgccatacgtcaaattgcagtgagtttcaccaaatcatggagatgttacccagt
tagcatacaactctttgcacaagtgcataatgtagtccctatgtcacaaggttatacgaagcatgtca
aatcatcgcctttagttacgatgtagttccacaagcgaaattagtttccgaaatggtcaagcatccaa
gtttagctcgaatctttaaggagatactcgaagtgcctatattacggaggtattatcatgtagcaagc
gttacctagcttattagtccacgaatcatgtgttagaagtcgtcaagttcatgttatcctaccagCCG
CCCGTCTTGAAACACGGACCAAGGAGTCTAAC
Nucleic acid 2
(SEQ ID NO: 59)
TGATTACGTCCCTGCCCTTTGTACACACCGCCCGTCGCTAttactgatcgaacgtcgtataatgctga
ggcatctgttattaaccgtacctttcaaggattaccatgtggcaacataagtAAACTTGGTCATTTAG
AGGAAGTAAAAGTCGTAACAAGGTTTCCGTAGGTGAACCTGCGGAAGGATCAcatccttggtctaaga
aagtgcatgatttgagcataccaatcgccattacgataaagatcctttgagtctaacgtacactgtgt
catctgtaagataccattgtcactacttcagtcagaACTTTCAACAACGGATCTCTTGGCTTCCACAT
CGATGAAGAACGCAGCGAAATGCGATAAGTAATGTGAATTGCAGAATTCAGTGAATCATCGAATCTTT
GAACGCAACTTGCGCCCTTTGGTATTCCGAAGGGCATGCCTGTTTGAGAGcattgaacacttcgtaag
gtacacctatggatcaacgattaagtctcgataccgtaagatggtaactctagtcagtgataatcaac
agcgtagtacattcgtaagcagtcttggacattactttctgagtgcaacattcaacgtctaaacgggt
taaatctctcataacggaacttgtgtgcaacagatgctatatggtatgcaaatgcgatacactttgAC
CCGCTGAACTTAAGCATATCAATAAGCGGAGGAAAAGAAACTAACgtaaagctattaaccggagtgaa
tccttcattaaagtcgcacaagctgtattaccgttacgcaacgtatttgattgaccatgtgaacagaa
gtaccctattgacctagattatgcagcaatgcctaagactatttgcctaattcgggctatttagacca
atcctccatgatgtatatcagtcaaggctagtttggaacatacacgaaagtccttatgtagtagagtg
caattctcgtatccttcaacagtgttatcgagtatcgaacgattatcctatgggtatccacttataga
acgtgtgtagactaacctgtaaacgatgtctctgaaagcaagactacttatctgagatcggatgttta
agacgctatgacaccattaacttatgccagtgctagtcattatgaccacgatttggaatttatggcta
tcgccactatgaaatgctaagctacctgaacaatttgtacgcagtgacagtagatcctttgatccaga
acttattaagagctgaccctatgaaacgtgatgtcctattcattattacgggaaaccgtagCGACCCG
TCTTGAAACACGGACCAAGGAGTCTAAC
Nucleic acid 3
(SEQ ID NO: 60)
TGATTACGTCCCTGCCCTTTGTACACACCGCCCGTCGCTAttggccttcagtcgagaacttgttgaaa
ctgtcctgacgcactggaacgagcttccattgattcgctagaaatgccgaccAAACTTGGTCATTTAG
AGGAACTAAAAGTCGTAACAAGGTTTCCGTAGGTGAACCTGCGGAAGGATCATTAcacagtgtggatc
tgacgaattaccaaggcactccatgtgtgccatctacgtctcaggaattgtacctgctaccactaggc
atcgagaacgctgcatgtattcaccgagtaaggtcttccagactccgataccgtatgtgttcccagga
gaaatgtcgcttagccggttcaagccatcatgtgctagactagacacgtctatcgcggtttacacgac
catcagttgagccaatgctatccttgcgggtcaaacagagcttacggatcacccatagttgtcacgcc
acgttaaagttccgagcgaaacgctatctcttcgagagctgtcccaatgaaactctgcacggacttgt
attgcacAAACTTTCAACAACGGATCTCTTGGTTCTCGCATCGATGAAGAACGCAGCGAAATGCGATA
CGTAATGTGAATTGCAGAATTCCGTGAATCATCGAATCTTTGAACGCACATTGCGCCCCTTGGTATTC
CAGGGGGCATGCCTGTTTGAGCGTCATTTactatgaggcccacagttacgaacgactagaccactgtc
ttacgagtgtcgcaccataagatggcgagtaatccgctcaatccactggttcctgagaaagagccgga
aatctgaggtcattctgcccatgatagctggaaacacccgagtctctaagtgtgagtagcctgatcta
ctgcaaacgcccgatacatatcgtgagagtctgctaggactgatcGTTTGACCTCAAATCAGGTAGGA
GTACCCGCTGAACTTAAGCATATCAATAAGCGGAGGAAAAGAAACCAACtcaggctatattgaggcac
cgcctggctagtagattacgacagctataacttcgggcaagccggttgatccaactatcgaaacctcg
ttagagcagtgtgtggcctaatggcatactggaacctatctgttacgccgagaactcgtgagcaactc
agtctcataaagtcatggtccgcactgatgctgcacaaagctaccgattgatacgttcgccgactgtg
atgcgtgaatcattccgtcaaagtgtccacccgtgtaggcattggtatatcgaccgatccaagaagcg
acgcttagtacgcgattacattgggcagatggtacagctcccataaacgctaggaactgttcgcaaga
gtcctgtgtcagagtcaaggataccgttcagaggcaaactgaccgtcattcgtgctaaacgatgtgat
ccgccctttcagacgctagtgttacctggaagaagattggcgctacctatgtcccatacagcgacaag
gtcttgtagaaggcatgtcaagctccctaaatggctccgctaaagtacgtgttgagggtctccaaCCG
CCCGTCTTGAAACACGGACCAAGGAGTCTAACaaaCACCGGCTAACTCCGTGCCAGCAGCCGCGGTAA
TACGGAGGGTGCAAGCGTTAATCGGAATTACTGGGCGTAAAGCGCACGCAGGCGGTTataagagcttt
gagcccacccgcatactgatttgactgccttaacttggtgaagccctcggacggaaacttgacatctc
gttctatctgaatgagcgcggcacagcttgagtctacttggaattgcattagcaccggcctgccttac
aacactgttgcgtattggactaactagcggcctGTGGGGAGCAAACAGGATTAGATACCCTGGTAGTC
CACGCCGTAAACGAT
Nucleic acid 4
(SEQ ID NO: 61)
TGATTACGTCCCTGCCCTTTGTACACACCGCCCGTCGCTAcctagaaagctcgccattagccgcagta
gtgattggacatcagagtttcgctcacaacgtcaccgctcgttatggaacttAAACTTGGTCATTTAG
AGGAACTAAAAGTCGTAACAAGGTTTCCGTAGGTGAACCTGCGGAAGGATCATTAaagcgttggttcg
ttacgcaaggctctacgaaagcagtgtctacttagcgttcagtgcagcgatccacaatctcatgggta
tgtcatcgaccagctacgacgcaagtttcccagatcaagattaggtgcccttcaagcacggttggaac
tctaccgacaattacgaggtcccaattacgggggcaactatgctgtaccagtaagatcctgccgattc
gacgcacagtcataactcagtgtacgtgtatcctggcaaggaggaagctccctttacatgctagtgca
atgtccgcagtttgcgagaggactatatccagtctaccacaggtcagaggttacaccctggctatcta
gtatggAAACTTTCAACAACGGATCTCTTGGTTCTCGCATCGATGAAGAACGCAGCGAAATGCGATAC
GTAATGTGAATTGCAGAATTCCGTGAATCATCGAATCTTTGAACGCACATTGCGCCCCTTGGTATTCC
AGGGGGCATGCCTGTTTGAGCGTCATTTaccgtaaagctaggtcaggtcttcactgggcaacgacata
atgggtaactcacttccagcctacatcagcggtgtcaaaggtagatgcctatcgtaccacccacaatg
ctctagggtttcagagaagctgtgtcttccgatggtcaccagatggattcgactcaaggtcatacagg
agtgtcgcgtaacatagcctatgcaaccgttcggttaaggacgtGTTTGACCTCAAATCAGGTAGGAG
TACCCGCTGAACTTAAGCATATCAATAAGCGGAGGAAAAGAAACCAACgctgcttagcctataccgta
atcggtgtgcgtgaacactagccaggtactgaatctaggatcgctgtggatctaaccagtccgctacg
acaagagtttactaggaccgcctaaatcatcggcgcttaccgttaagaaacctgtccggcgacatata
cagtgccattgcgcttgagaatcatgctgtgcgagagacatacacggttccgagttgacatctacgtg
aagggcatctttcgatgctgacccgaagtttatctgggaagctacgtcatttgcctaccgctgcgact
aatctttgcagacgacatgctatgagcttgctggaccacgaatcgttaccagtcatctgagacacttg
gcatacgcttgggcttgatacacctatggatgggatacactgatcggctgccgcataatttgctacgc
cttacagagaagtgcagtctaccggctgttaatactccggctttacacgagaagctactgagggccat
ttgacacaatcgcgtgagtttgctgatctgacatgggctgaaacatgagcctccgaactatcgtCCGC
CCGTCTTGAAACACGGACCAAGGAGTCTAACaaaCACCGGCTAACTCCGTGCCAGCAGCCGCGGTAAT
ACGGAGGGTGCAAGCGTTAATCGGAATTACTGGGCGTAAAGCGCACGCAGGCGGTTgtagttaggcaa
ctctaggcggcaactgctcatcaactaggagtacagtcaatctgacggacgcgctactgcatacttag
tcatctactggttccagagccacgggtcatcgtaaattgggtattccgaaatggcccacacgccgttc
acgtttcaaatgattggcatctagggacacctGTGGGGAGCAAACAGGATTAGATACCCTGGTAGTCC
ACGCCGTAAACGAT
Nucleic acid 5
(SEQ ID NO: 62)
TGATTACGTCCCTGCCCTTTGTACACACCGCCCGTCGCTAtcaggaagtgtgtcccattgccggagga
gtcctattgaatcacggattacgtctgtaacgctggaccgaggttgtatcatAAACTTGGTCATTTAG
AGGAACTAAAAGTCGTAACAAGGTTTCCGTAGGTGAACCTGCGGAAGGATCATTAgcttcgattacga
tgcccaaatacgatccgcgtagtttccacgaggtctacagtaccctattgttcgaggcagtaacctga
accgcgtctgtcaacagttatgtgacggcaagttgtccaagtccgagccatactatcagtcgtcttag
ctcatgggaagctcgcagtgttaagctcagtaggcaaattccagcgtgatgccgatccagtgtacgag
aatccttacatgcaagtgtcgcaggccagatcagtttcgagaaagagtacgttctatccctggcgtcc
tcagtgactcaagatgagattacatccacacggtctcggtccattcgcaaagtacagtgtttccttag
cagcaggAAACTTTCAACAACGGATCTCTTGGTTCTCGCATCGATGAAGAACGCAGCGAAATGCGATA
CGTAATGTGAATTGCAGAATTCCGTGAATCATCGAATCTTTGAACGCACATTGCGCCCCTTGGTATTC
CAGGGGGCATGCCTGTTTGAGCGTCATTTaacatgctgcgtagtacgtcgatcaccaagctatgagcg
ttgtcaaaggagtgtcaaccgacgagtccaggtttcatcaccttgctaggtatccacaggtgcattag
gcggctaagtcttccacatcgtattgccgaagtgtatcgcccagacattcaagctgtcagaactctgc
gttacagaacgtgccgtcaagattcaggctatcatccgtgaaccaGTTTGACCTCAAATCAGGTAGGA
GTACCCGCTGAACTTAAGCATATCAATAAGCGGAGGAAAAGAAACCAACtacgtgagatcggtccgat
atgagctgtccacaatagccatagactaggagtcacccttcgagtggttctagcacatccagatgaca
cactaagtgccctgttcgggacttgtaaagcacgattccttggttaagacgcctcccagtcagtatca
tggtcgtaaagttcgtccagtggtcaacgctcttcgtcaagcgataagttaaagccggtagctgctca
agcctgccatacggattagttcaaacgagcctgtcgtgtacgttctccgcacaatgtctaacaatggt
acggtgcagatagcttccgcccaggttattaaggcaaattggcccatccattctgtcggtcggcaaac
agttcctgaaattccgctgaggttgtaagacccggtctgaatagccagatcaatacgtcggtgctgat
gagtgccatcacagtttctctaggatagcgcacgttcatgtcgcgtaacgcatctagcatttaggtgc
aacggtactacgtccaccagtaggaagttcgcataaacggtcaccttagcctgagtagccgtcaaCCG
CCCGTCTTGAAACACGGACCAAGGAGTCTAAC
Nucleic acid 6
(SEQ ID NO: 63)
TGATTACGTCCCTGCCCTTTGTACACACCGCCCGTCGCTAtcccgcaaatacctttggagtgcgtcac
tatctaggagtgtgccgatgactcgtaatctccatcctcgaagttgcacgatAAACTTGGTCATTTAG
AGGAACTAAAAGTCGTAACAAGGTTTCCGTAGGTGAACCTGCGGAAGGATCATTAataatccagggtc
cacgagtgaatgccctgcaaatgtaccaagttcctgaccttctggcatgtgaagccgatcttatcgct
gaagagtctcgaagtcgctgacatacacccgtattgtcgatctgttggcgtaacggacatacgatgca
ctgacagcagttgcttagagcctagacacgacattgccttgaacgaccttgctactcatagggatacc
cgacgtagacgtttagtcctgcaagtcgaaagccctttgtgagagtcgccttatagtaccggatagtc
tcccagccatattggagagtccatatagccacggtagaatgctccgaggtaacctgagtcaaattgcc
gcactagAAACTTTCAACAACGGATCTCTTGGTTCTCGCATCGATGAAGAACGCAGCGAAATGCGATA
CGTAATGTGAATTGCAGAATTCCGTGAATCATCGAATCTTTGAACGCACATTGCGCCCCTTGGTATTC
CAGGGGGCATGCCTGTTTGAGCGTCATTTagtgacagttcacggtagcagctaaatcttcgggcatca
cgagtacatgagtctcccatcgttaatccagcaagccgatgtggagctatttcaacgggacgtatatg
tcgtccatccgagttgcggactatctacagggtgaattatgcgactgactgccttgccactacgaaac
agtgcgttcaaattgcgctaagggcgtgcgaatacttatgcaggcGTTTGACCTCAAATCAGGTAGGA
GTACCCGCTGAACTTAAGCATATCAATAAGCGGAGGAAAAGAAACCAACatgtccaaccgaaactcgt
gatcttagtgaccgcacggatctgtcattcgagaagcgtagagacttatgcctgggccttaacttgtg
ctcagtagcctcaagagaactgcctcctgtctattacgggtaaactcctggtgatccagagacgtagt
gtcagaacagcctagatgtgttgccacgacctgtaaacggctttcttacgacgcaatgctgatggtga
ctggcgattaacgaaccgaatcatcctgtgtgcatcctacggtgtgccatttgaaccagagagtatct
tcgaccacgatctgcaagggtgtcatgcttgacctagagtaccacgttcagttgcctcatagggctta
gcagcgtattcatgcgacttgcgataacgatgtcctgtacggacgttccatagtccgacaaacccatg
tatgtctgcgagaggttagccaagagtgcttactccacctagtgagatgtagcgacaacgactgtgag
tgtacgactccttagggtatagcgttgccaaacttcccaaggtagggagcctttcccattacgaaCCG
CCCGTCTTGAAACACGGACCAAGGAGTCTAAC
Nucleic acid 7
(SEQ ID NO: 64)
TGATTACGTCCCTGCCCTTTGTACACACCGCCCGTCGCTAgacaccctgttcagattagcgagcctca
gttacaccagattccgagttcgtaagatcgagaggagccatcatggacgtttAAACTTGGTCATTTAG
AGGAACTAAAAGTCGTAACAAGGTTTCCGTAGGTGAACCTGCGGAAGGATCATTActgacggaccaat
ctgtatgtaaagcggctattcaggagcctatccgacgagttgatgcttacaaggcgatctatccctga
ccagtgctaaccatgtgcataagagcagtctcactcacgagtctcggttccttagacgattcaatgcc
aagttgtgccggagaacacctgttgatcctcgacaatgattcagtccaccgggatgtctgtagttccc
aacgccaatatgtagagcttcggtccacgaaagtaccgtggtagccatgatatgacttacgcccgaca
aagttcgggagtttctcgcatgtgaagtttccgcaaccatgagcaaggtcgtttgacctggaagtgta
tgatccgAAACTTTCAACAACGGATCTCTTGGTTCTCGCATCGATGAAGAACGCAGCGAAATGCGATA
CGTAATGTGAATTGCAGAATTCCGTGAATCATCGAATCTTTGAACGCACATTGCGCCCCTTGGTATTC
CAGGGGGCATGCCTGTTTGAGCGTCATTTatctgacagccttctacgagcctgctgaatcagatgaac
cacttggtcgcaatgatcgcaaggtcgggtatatcttcacggttagatccgaactgctccactgggta
caacacactgacttggtaactcggtcatacacgtcgggaacataactgcctgtgatagcacgcactct
taggacagtcgcattctctaggtcatggaatagcgcaacatcgctGTTTGACCTCAAATCAGGTAGGA
GTACCCGCTGAACTTAAGCATATCAATAAGCGGAGGAAAAGAAACCAACtccacagtatcatccgatg
gagcgattcgcatacgacagtcaatggctattggtcaggacctagcttccaagtcaagggaaggtttc
aggatcgtcgcatcgtactttcctacgaagtgcctaaagggatcactctccgaacggtttgtatcagc
gtgcagatgtacctgttacgccagaggaatgacattctacccgagggatcttacagtccgggatttgt
gcaatcacagttgggctctaacgtcaagcgaggtgtatgtcccatgaataaggacggctttctcaggc
caagaagtctacgcagaagttacccagctcgtttacggtgtccactcaaagtctagcatgttccggtg
acctagttgatggcagtagcagtaccatgacaagaggcttccgattatccagacccagttgtgggcta
atatgagcagcaccctagtatttcgcgcaatgccggttatatgaaggccacgtacaagtttctccgcg
catgtgtcagatagtatccggttccacagcataagtccgccagttggttcactaagttgccgacaCCG
CCCGTCTTGAAACACGGACCAAGGAGTCTAAC
Nucleic acid 8
(SEQ ID NO: 65)
TGATTACGTCCCTGCCCTTTGTACACACCGCCCGTCGCTAcatgactggaaaccctctgacgtgtaac
tctggaagctcagttatcggaaacggcgctaagctacgtgatcgtaagcagtAAACTTGGTCATTTAG
AGGAACTAAAAGTCGTAACAAGGTTTCCGTAGGTGAACCTGCGGAAGGATCATTActctgatggacct
ggtgatacacggtactatttggcatggtcacatcgggcatctgtaagacctccagttgtagtgtgcag
agttcccagacagtctaagacggcattgactatggccttgtggttcgagaaccgaacatccaagagtt
tcgctcgttcatggcgataacccttcaacgtgtggtaacctgtaacgcagtcagctttagcgcgtgaa
taccttgaggcaatacaccgagttgtgctaccctagtgatgacagaatggcaccttatgctccggtac
acctacggaatcatgcaagtggaatccctttcgagagcaggctcagtttagttgcgaagtgatctccg
catttccAAACTTTCAACAACGGATCTCTTGGTTCTCGCATCGATGAAGAACGCAGCGAAATGCGATA
ACGTATGTGAATTGCAGAATTCCGTGAATCATCGAATCTTTGAACGCACATTGCGCCCCTTGGTATTC
CAGGGGGCATGCCTGTTTGAGCGTCATTTaacttagggagtatgccgtcgaacatcgctcgtgagtaa
cttatcgtgcggatacacctcgtacatgccactcggtacttagaatagctggtaacctccgatgctcg
caatgcgtagttctggattccaatggaccaacggtcattcctgggtgacaaagcaatctcctgtagca
ggtcacagttctcgtctcgcagtaacgaagtcctcttacgtcatgGTTTGACCTCAAATCAGGTAGGA
GTACCCGCTGAACTTAAGCATATCAATAAGCGGAGGAAAAGAAACCAACtattgacgaccgttgccag
agagccatcacttggtttcgactataacgacagatccgtggcctcctaaagttgcgtatgcagtatcg
agatgtaccctgcgaaccgagtgtactaacgtgtctgaggaatccattcccgtatcgggcacaacagt
atgtgtcttccagatagagggcctttgctgacgaagtcctagactatcgcttagagacgcctacagac
cagtaatcgtgaccttctacctgagatgccgtgaacataggtgctaatccgagagcatgtgtacgaac
tccgaaccttgccattaagggatgagcctactgaactaccgctgatcgtgcgagtatatcctgctgct
aacgtaaactcctgagggctacagctaaacagcttggacctagtgtcatatcgccgttccaactgact
ccttgagagactgcgtaagatttccgccgacattgccaaacgctaattgccgatggtgtaaacgaccc
gcattccattggttgctaaagcctcgtaagaatccgggctgactatcatgtgagcttgacgctacCCG
CCCGTCTTGAAACACGGACCAAGGAGTCTAAC
Nucleic acid 9
(SEQ ID NO: 66)
TGATTACGTCCCTGCCCTTTGTACACACCGCCCGTCGCTAgcacctagcctttaacgagaagaatgta
gccctacgccatcggcatgtgattccatacgatgttacgaaacctgaggcagAAACTTGGTCATTTAG
AGGAAGTAAAAGTCGTAACAAGGTTTCCGTAGGTGAACCTGCGGAAGGATCActtctgaaactatgac
gcgccaaccggaatcgtgtaatggattgacctacttgctcggacgacggataacgctgtatgcaaatg
tgcctgtaactcggctctgcgaactgctctgatctaACTTTCAACAACGGATCTCTTGGCTTCCACAT
CGATGAAGAACGCAGCGAAATGCGATAAGTAATGTGAATTGCAGAATTCAGTGAATCATCGAATCTTT
GAACGCAACTTGCGCCCTTTGGTATTCCGAAGGGCATGCCTGTTTGAGAGtccacgtaaatcagcgcg
ttatgggtctgacgtaagcacaagggtcctatacacgctactctggttatccctgagaagtcggttac
catgtcacacagtcaggctatatgccctcacgttgattcgagcgaagttactgcaccaagtctggcgt
agttagtgttccgtagagcaagtcactcaatcccgagcaaagtgtcgtgatgctgttcagcaagacAC
CCGCTGAACTTAAGCATATCAATAAGCGGAGGAAAAGAAACTAACaggtcctcagaggctaatgtttc
atgcaatgagatcccgcgtggacaccaccaagattctactgttgtcaagatacgggcgactcgacatg
gagctactattctatcagaagagccctgccaggcgttcaatcgcatttccatttaatggctgactcgc
gcagacgaagtctcctagagttaagtcttacgagcaccgcttgtgtgagcacgatcatacgatactga
ctaaggcgtcaccgagtttcagaccctacgacatgactgtctttaggccagagtctactagaccgagc
tttggatgccaacctttccgaagtgagatttacccacagcgttcgtgtgttcgactaacccgcaaagt
gttaccataggctggtcctatttcgcagtggctagagagcaatgttccaggatgtgctactacttgcc
gtgagctagacataccgatggctaagtggatacgttacaggcgcacgtagttctaaccggcttatacg
gataacctgacccgagcgttattcttatgccgcagagaggtttcttacccgaaggcactagCGACCCG
TCTTGAAACACGGACCAAGGAGTCTAAC
Nucleic acid 10
(SEQ ID NO: 67)
TGATTACGTCCCTGCCCTTTGTACACACCGCCCGTCGCTAtgcggagcatcctagtacaatatccggt
tgcctataagcccggtatgcgcgaattaacctaactgccagagatgagttccAAACTTGGTCATTTAG
AGGAAGTAAAAGTCGTAACAAGGTTTCCGTAGGTGAACCTGCGGAAGGATCAtaggtcacgctagtac
caaggagactcagaccttacagcttgcttgcagacagatcggaatcccacagcagagtttagacgttt
ggagacagtcccacttcagtcgttggatgcacttagACTTTCAACAACGGATCTCTTGGCTTCCACAT
CGATGAAGAACGCAGCGAAATGCGATAAGTAATGTGAATTGCAGAATTCAGTGAATCATCGAATCTTT
GAACGCAACTTGCGCCCTTTGGTATTCCGAAGGGCATGCCTGTTTGAGAGcagggttccctagtaagt
acgattccaatacgcgatccgaatgcggcgtttcctaagcaaggtataatctcctgacgaggagtcgg
gtccataaggtttccatagttcaccgtgagactgcgatggtctgccaatgttcacttcaagtccgtaa
gacacggcaagagcctagcatctgttcgttcagagtcatggtatcggacaactgcctgatcttcgaAC
CCGCTGAACTTAAGCATATCAATAAGCGGAGGAAAAGAAACTAACgtcacatgcaagctgtttccttc
tacatgacgagcctctgcgataggtgagtatcccactcattgatagctgccgcaagtcaggagaatac
gtccgttagtaaactgtcccatgccgaagctcaagacctggaagtccttgataactggcacactctga
gccaactgaacgtgtacgcattacaactccggtgttagcctgcttagctgaaccagcagtaattgtta
ggcgtcccaacgatccatgatccgcgtgaagaaatctttagcgcccataggcagtaaggtagcccgac
atagtgtctattaggcccgaaatcccttagggagcccaatacatgatcttagccgagtcgtaggaacg
tccatctcgaaagtcgtttgctagggcaatccaagtctcgatcccgataagttctggctaggttgaca
aagcgtccagatccgacgagtaaatggtccctgttaatccgatagtcgcgcaccacggtgaatatagt
ccgatgacattgacctgtaccagaccgcgtctcaaattgacgaaagcgatgttcgtaaccgCGACCCG
TCTTGAAACACGGACCAAGGAGTCTAAC
Nucleic acid 11
(SEQ ID NO: 68)
TGATTACGTCCCTGCCCTTTGTACACACCGCCCGTCGCTAacggcactgatgttcacccgccgtcgat
catacacgcagggcgatgactctatgcgaggctccgaccagtaacaggcgctAAACTTGGTCATTTAG
AGGAACTAAAAGTCGTAACAAGGTTTCCGTAGGTGAACCTGCGGAAGGATCATTAcctggcgaatgtc
taaggcgtccatatccgaggtgcagcgcgttgcctgaccattaggcccgtatagttcggcgtgaccga
gatgccgctcagtacgacggtctaacaagctggccgcacttgccaacctgtcgcggactgtcttaacg
gtggcccgacttgctaccacacccgtgggattgtgctacgaagcgtcccgaaggtcctcagcccaaga
gtcctgtagtgagtacccggagcctcgaccctgatgtgatccgaccagattggagccggtgaccctca
gacggagtcaaggtcctacctgtgaagccctgacggcgtggattcctgctagagccaaggagagtgtc
ccgctacAAACTTTCAACAACGGATCTCTTGGTTCTCGCATCGATGAAGAACGCAGCGAAATGCGATA
CGTAATGTGAATTGCAGAATTCCGTGAATCATCGAATCTTTGAACGCACATTGCGCCCCTTGGTATTC
CAGGGGGCATGCCTGTTTGAGCGTCATTTgcggacgatgcctttgtcgataatgctcccgctgtaggc
cagcgccaatcggctgtgcatttagcgaggtctcacgccagtgcgagtacgagccttcctcctaagcg
ttcggtcggacaggacatctggatcgcggaaccctaatcccgtgggacaccgtcacttggtcgatgcg
cgtagcttgtcaccgcagggactgagaggtcaacccatgcgactgGTTTGACCTCAAATCAGGTAGGA
GTACCCGCTGAACTTAAGCATATCAATAAGCGGAGGAAAAGAAACCAACggtggaaagctcgtctccc
aatgccattagcctcggcggagcgatagcagctcctctggaagcatcagtgcgtctgcccaaggcgtt
cctcgtcggtacaacgtagactgccgctacggacggtgtcaccagggatacactccatagcatccggg
tcgcaaggtgtgcgtgccaactacccgacttctaacagggctggccgatactgcgggctcaagtgact
cagatcctgaagggcgcaccacgtcgcggactacagtgttcacatgaagcgcggtcgtgcagcgcatg
gtccataccaactgcctagtacgcgggactggcgtcgaatcgactcgtccttcggaaacatgacggcg
cggcctaagcgagaactctgctcgtgtccatcaacggctggcggcgatatgtcctgacctcagccata
gtgcctacctcgggagcgttcaagcgatcctcggtcttaacgggcgaactcgggctcgaaagcgaatg
cctccctaagctcttcggtggcggacgcggaatcatagctcagcgaactctcacggttgcaggcgCCG
CCCGTCTTGAAACACGGACCAAGGAGTCTAAC
Nucleic acid 12
(SEQ ID NO: 69)
TGATTACGTCCCTGCCCTTTGTACACACCGCCCGTCGCTAcgtacctgtcagcacgctgttgacctta
gcccgtggcaacgactgtgaagcctccgacacgtactgagggcgattcccagAAACTTGGTCATTTAG
AGGAAGTAAAAGTCGTAACAAGGTTTCCGTAGGTGAACCTGCGGAAGGATCAccatactgcgaatggg
agccgccggaggtaagtcctttccctgatgaccttgcgcgtagggccgggtaagagcttctccactga
ctgtcaaccgtgggcacgccgaggatgctactcatgACTTTCAACAACGGATCTCTTGGCTTCCACAT
CGATGAAGAACGCAGCGAAATGCGATAAGTAATGTGAATTGCAGAATTCAGTGAATCATCGAATCTTT
GAACGCAACTTGCGCCCTTTGGTATTCCGAAGGGCATGCCTGTTTGAGAGggcagctttacggttccc
agtgcctaatgaggacgcctgggcggaatcgagccttcggaaagacatctgcagcacggtgcctgcaa
cctgtcggtgacgtatcaggacctggtgtccacccgttgtcagggcttccaaggtcaagcaagtggtg
accggccatgcgtggtcgcttcacagaacatcacggcagtcgccgtatcggcccgagtgagactagAC
CCGCTGAACTTAAGCATATCAATAAGCGGAGGAAAAGAAACTAACgtcgtgacacgcttcgacgattg
agtcgccgcctacgactgacgatcttccgcctgtagctggatgtgcccgatccgtgaggacattccca
cctggactgactcgcatggagactgccacggtgattcgcaacagcccgtagaggcttcgttcgaccac
ccgatgctgaaagctgctgcgctgatctgagacctcggagggcgtaaactggacacctgccactcgga
ctgtgttcgcacgtcggcttcatagccactggcaaccgcgcttgtgtgcagacggaaccctttagtgc
ctggcgatgaccctactcccggtgaacggcaatgcaatgggcctggaactgtgacgctcccgtacctt
cccttgagaggacctggcatctggacgcaactcctgggtgtgacctgtgagcaacgcctcctactggg
tatagcccgcgcttagacgctgctagagccggagacatacgatccctgcgcttacacgcacgcgatag
gtgcgctcgataatctcggcccggtagtgcaacctgaccagcggtagaccttgatgacggcCGACCCG
TCTTGAAACACGGACCAAGGAGTCTAAC
Nucleic acid 13
(SEQ ID NO: 70)
AACTTTCAACAACGGATCTCTTGGTTCTCGCATCGATGAAGAACGCAGCGAAATGCGATACGTAATGT
GAATTGCAGAATTCCGTGAATCATCGAATCTTTGAACGCACATTGCGCCCCTTGGTATTCCAGGGGGC
ATGCCTGTTTGAGCGTCATTTgtcgggcgactgctctcatgaccagcgtgggcgtccatggctgagcc
tcgtgtggctcgagccgacgtctggccgtgagctcgggagggctggtcgagctgctgccacgctctcg
gctcgatcaccgtgtgacgtcggcgactccaccacggcacggcgacggtgtcacgcgctcctgggGTT
TGACCTCAAATCAGGTAGGAGTACCCGCTGAACTTAAGCATATCAATAAGCGGAGGAAAAGAAACCAA
C
Nucleic acid 14
(SEQ ID NO: 71)
AACTTTCAACAACGGATCTCTTGGTTCTCGCATCGATGAAGAACGCAGCGAAATGCGATACGTAATGT
GAATTGCAGAATTCCGTGAATCATCGAATCTTTGAACGCACATTGCGCCCCTTGGTATTCCAGGGGGC
ATGCCTGTTTGAGCGTCATTTcccaggagcgcgtgacaccgtcgccgtgccgtggtggagtcgccgac
gtcacacggtgatcgagccgagagcgtggcagcatttatattgcaatataaatgctgccacgctctcg
gctcgatcaccgtgtgacgtcggcgactccaccacggcacggcgacggtgtcacgcgctcctgggGTT
TGACCTCAAATCAGGTAGGAGTACCCGCTGAACTTAAGCATATCAATAAGCGGAGGAAAAGAAACCAA
C
Nucleic acid 15
(SEQ ID NO: 72)
AACTTTCAACAACGGATCTCTTGGTTCTCGCATCGATGAAGAACGCAGCGAAATGCGATACGTAATGT
GAATTGCAGAATTCCGTGAATCATCGAATCTTTGAACGCACATTGCGCCCCTTGGTATTCCAGGGGGC
ATGCCTGTTTGAGCGTCATTTtaaggcccatgttgtaggtcgaattgctagcaattcgacctacaaca
tgggccttaatgctgtgcgcaccaagaggatcaaccagtgtcggatgcatccgacactggttgatcct
cttggtgcgcacagcatttacccagaagtgtattcctcgaggaatacacttctgggtaagcgtagGTT
TGACCTCAAATCAGGTAGGAGTACCCGCTGAACTTAAGCATATCAATAAGCGGAGGAAAAGAAACCAA
C
Nucleic acid 16
(SEQ ID NO: 73)
AACTTTCAACAACGGATCTCTTGGTTCTCGCATCGATGAAGAACGCAGCGAAATGCGATACGTAATGT
GAATTGCAGAATTCCGTGAATCATCGAATCTTTGAACGCACATTGCGCCCCTTGGTATTCCAGGGGGC
ATGCCTGTTTGAGCGTCATTTgtggtggagtcgccgacgtcacacggtgatcgagccgagagcgtggc
agcatttatattgcaatataaatgctgccacgctctcggctcgatcaccgtgtgacgtcggcgactcc
accacggcacggcgacggtgtcacgcgctcctgggttaccgcggctagttcggcgtggctggcacGTT
TGACCTCAAATCAGGTAGGAGTACCCGCTGAACTTAAGCATATCAATAAGCGGAGGAAAAGAAACCAA
C
Nucleic acid 17
(SEQ ID NO: 74)
AACTTTCAACAACGGATCTCTTGGTTCTCGCATCGATGAAGAACGCAGCGAAATGCGATACGTAATGT
GAATTGCAGAATTCCGTGAATCATCGAATCTTTGAACGCACATTGCGCCCCTTGGTATTCCAGGGGGC
ATGCCTGTTTGAGCGTCATTTggggcggttaaggaaagtcaaactcccgggctgtgaaggcccagtag
gttgcgtagctaagacagcacctcataggcatgctgtgcgcaccaagaggatcatgcctatgaggtgc
tgtcttagctacgcaacctactgggcctaccaagagacgttacccgttaccgcggcggctggcacGTT
TGACCTCAAATCAGGTAGGAGTACCCGCTGAACTTAAGCATATCAATAAGCGGAGGAAAAGAAACCAA
C

The synthesis of the nucleic acids 1 to 17 was outsourced to GenScript Japan Inc. As a result, the nucleic acids 1 to 13 were synthesized, whereas the nucleic acids 14 to 17 could not be synthesized in the time required to synthesize the nucleic acid 13. This indicated that randomly designed, non-naturally occurring artificial sequences may include sequences that are difficult to synthesize.

Next, PCR was performed using the following universal primers and the nucleic acids 1 to 13 as templates. The universal primers used were for the eukaryotic 18S rRNA V9 region, the eukaryotic ITS1 region, the eukaryotic ITS2 region, the eukaryotic 25-28S rRNA D1-D2 region, or the prokaryotic 16S rRNA V4 region.

TABLE 1
Universal primer set for eukaryotic 18S
rRNA V9 region
[Table 1]

| | | SEQ |
|---|---|---|
| | | ID |
| Name | Nucleotide sequence | NO |
| 18SV9f | GTACACACCGCCCGTC | 75 |
| 18SV9r | GATCCTTCYGCAGGTTCACCTAC | 76 |

TABLE 2
Universal primer set for eukaryotic
ITS1 region
[Table 2]

| | | SEQ |
|---|---|---|
| | | ID |
| Name | Nucleotide sequence | NO |
| ITS1f | CTTGRTCATTTAGAGGAASTAA | 77 |
| ITS1r | GCTGCGTTCTTCATCGWTGY | 78 |

TABLE 3
Universal primer set for eukaryotic
ITS2 region
[Table 3]

| | | SEQ |
|---|---|---|
| | | ID |
| Name | Nucleotide sequence | NO |
| ITS2f | RCAWCGATGAAGAACGCAGC | 79 |
| ITS2r | TCCTCCGCTTATTGATATGC | 80 |

TABLE 4
Universal primer set for eukaryotic
25-28S rRNA D1-D2 region
[Table 4]

| | | SEQ |
|---|---|---|
| | | ID |
| Name | Nucleotide sequence | NO |
| LR0f | ACCCGCTGAACTTAAGC | 81 |
| LR3r | GGTCCGTGTTTCAAGACGG | 82 |

TABLE 5
Universal primer set for prokaryotic
16S rRNA V4 region
[Table 5]

| | | Sequence |
|---|---|---|
| Name | Nucleotide sequence | number |
| U515 | GTGYCAGCMGCCGCGGTAA | 83 |
| U806 | GGACTACNVGGGTWTCTAAT | 84 |

PCR reaction solution composition: 1×KAPA HiFi and 500 nM primer.

PCR reaction conditions: For ITS1/ITS2: 95° C. for 3 minutes; 95° C. for 30 seconds, 52° C. for 30 seconds, and 72° C. for 30 seconds, 25 cycles; and 72° C. for 5 minutes. For 25-28S rRNA DID2 and 18S rRNA V9: 95° C. for 3 minutes; 95° C. for 30 seconds, 57° C. for 30 seconds, and 72° C. for 30 seconds, 25 cycles; and 72° C. for 5 minutes. For 16S rRNA V4: 95° C. for 3 minutes; 95° C. for 30 seconds, 50° C. for 30 seconds, and 72° C. for 30 seconds, 25 cycles; and 72° C. for 5 minutes.

As a result, each region in the nucleic acids 1 to 12 was amplified with appropriate efficiency using the universal primers. On the other hand, the nucleic acid 13 was amplified with extremely low efficiency and was confirmed to be unsuitable as a standard nucleic acid.

### 2. Evaluation of Quantitative Properties of Nucleic Acids 1 to 12

Plasmids in which the nucleic acids 1 to 12 were integrated into a pUC19 vector were produced. These plasmids were linearized by cleaving with Bsal or BpmI, and then purified using AMpure XP (Agencourt). Concentrations were measured using the Qubit assay kit (Thermo Fisher SCIENTIFIC), and the copy number of nucleic acids was calculated. The concentrations were adjusted to prepare a mixed solution of plasmids containing the nucleic acids 1 to 12 (10 to 10⁶ copies for each nucleic acid).

A sample was prepared by adding DNA (1 ng), extracted from soil using FastDNA Spin Kit for Soil (MP Biomedicals), to the mixed solution, and PCR was performed using a universal primer set for the eukaryotic ITS1 region, a universal primer set for the eukaryotic 25-28S rRNA D1-D2 region, or a universal primer set for the prokaryotic 16S rRNA V4 region, to obtain an amplicon library.

PCR reaction solution composition: 1×KAPA HiFi and 500 nM primer.

PCR reaction conditions: For ITS1/ITS2: 95° C. for 3 minutes; 95° C. for 30 seconds, 52° C. for 30 seconds, and 72° C. for 30 seconds, 25 cycles; and 72° C. for 5 minutes. For 25-28S rRNA DID2 and 18S rRNA V9: 95° C. for 3 minutes; 95° C. for 30 seconds, 57° C. for 30 seconds, and 72° C. for 30 seconds, 25 cycles; and 72° C. for 5 minutes. For 16S rRNA V4: 95° C. for 3 minutes; 95° C. for 30 seconds, 50° C. for 30 seconds, and 72° C. for 30 seconds, 25 cycles; and 72° C. for 5 minutes.

The amplicons were sequenced using MiSeq (Illumina). The results were evaluated using a DADA2-based analysis pipeline, and quantitative results were calculated.

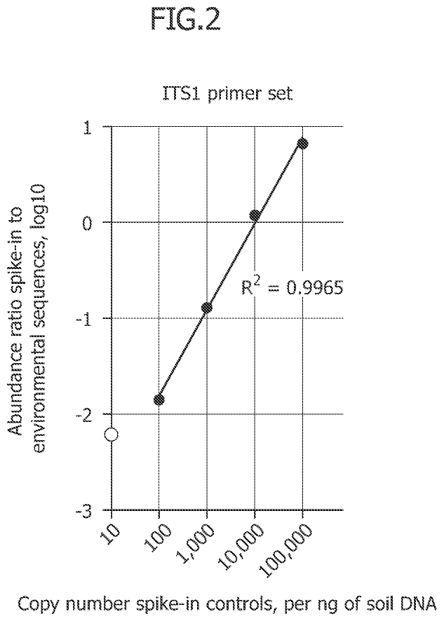

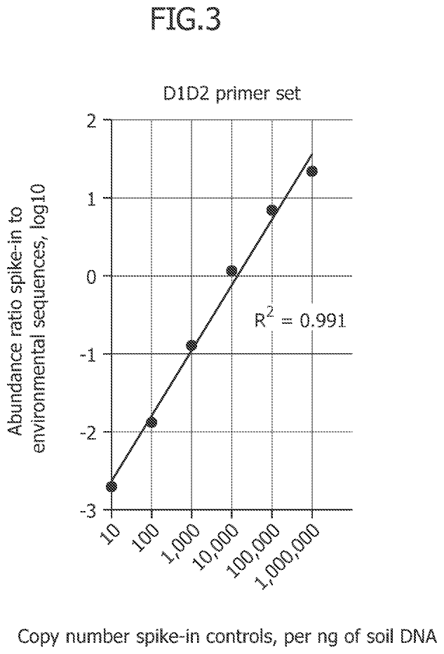

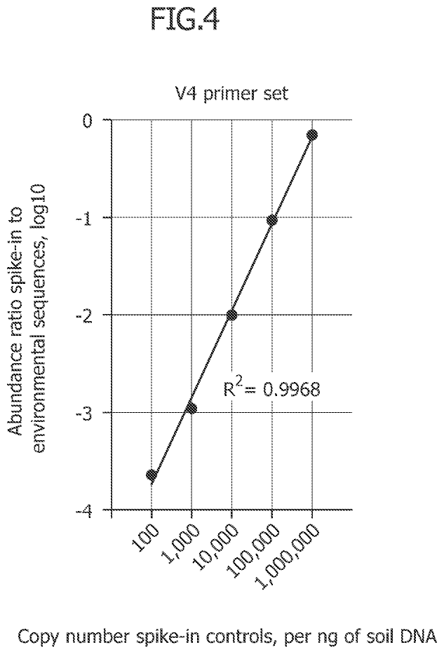

FIG. 2 shows the results using a universal primer set for the ITS1 region, FIG. 3 shows the results using a universal primer set for the 25-28S rRNA D1-D2 region, and FIG. 4 shows the results using a universal primer set for the 16S rRNA V4 region. The horizontal axis indicates the amount of the nucleic acids 1 to 12 added, and the vertical axis indicates the ratio of the number of reads derived from the nucleic acids 1 to 12 to the number of reads of the target sequence derived from DNA extracted from soil. In all cases of using any of the universal primer sets, it was possible to detect the nucleic acids 1 to 12 in an amount-dependent manner, and high quantification and linearity were confirmed. These results indicated that it is possible to verify the quantitative accuracy of metagenomic analysis using the nucleic acids 1 to 12.

### 3. Quantification of Fungi in Soil

DNA was extracted from samples in which mixtures of the nucleic acids 1 to 12 (4×10⁶ copies) were added to various amounts of soil (300, 150, 75, or 37.5 mg) using FastDNA Spin Kit for Soil (MP Biomedicals). PCR was performed in the same conditions as in 1 above using a universal primer set for the ITS1 region, so as to obtain an amplicon library for each sample. Amplicons were sequenced using MiSeq (Illumina), and the results were analyzed using the DADA2 pipeline.

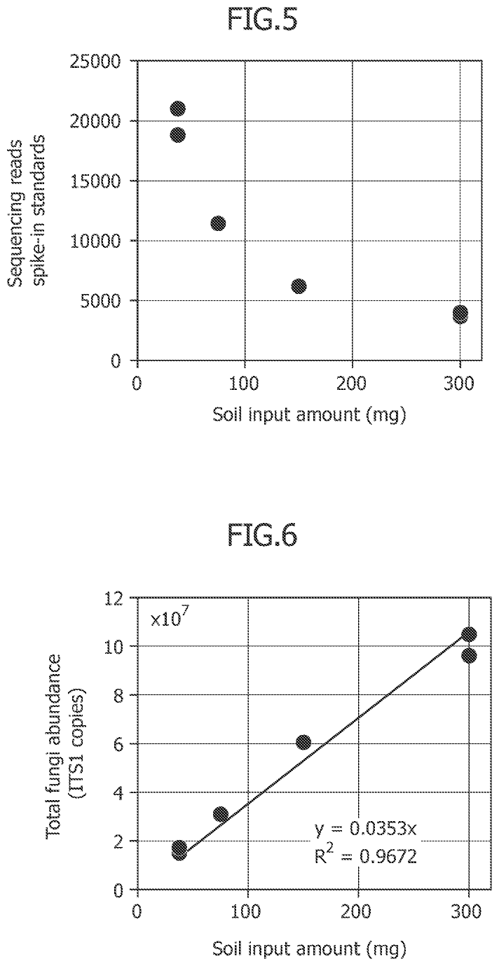

FIG. 5 shows the results. The horizontal axis indicates the amount of soil added to the sample, and the vertical axis indicates the number of reads derived from the nucleic acids 1 to 12 when the total number of reads in each sample is the same. As the soil volume increased, the theoretically expected number of reads for the internal standard genes decreased. Also, FIG. 6 shows the total amount of fungi estimated based on the number of reads derived from the nucleic acid 1 to 12. A correlation between the amount of soil and fungi was confirmed. These results confirmed that metagenomic analysis using the nucleic acids 1 to 12 as internal standard nucleic acids can accurately quantify the absolute amount of fungi in microflora samples.

### 4. Quantification of Fungi and Bacteria in Soil

Using authentic preparations in which genomic DNA of 10 types of fungi (Aspergillus oryzae, Candida glabrata, Candida tropicalis, Saccharomyces cerevisiae, Schizosaccharomyces pompe, Trichoderma reesei, Marasmius purpureostriatus Hongo, Hymenoscyphus varicosporoides Tubaki, Emericella nidulans, and Cryptococcus neoformans) and 14 types of bacteria (Clostridium acetobutylicum, Bacillus subtilis, Bacteroides vulgatus, Pseudomonas putida, Desulfitobacterium hafniense, Deinococcus grandis, Nitrosomonas europaea, Nitrobacter winogradskyi, Escherichia coli, Treponema bryantii, Gemmatimonas aurantiaca, Chloroflexus aurantiacus, Anaerolinea thermophila, and Desulfovibrio vulgaris) (fungi and bacteria were obtained from the Japan Collection of Microorganisms (JCM), RIKEN BioResource Research Center) mixed in known amounts, a solution containing 1.5×10⁵ copies of the fungal gene per 1 copy of the bacterial gene was prepared and serially diluted. The nucleic acids 3 to 10 (5×10⁴ copies each) were added to the diluted solution, and PCR was performed in the same conditions as in 1 above using a universal primer set for the prokaryotic 16S rRNA V4 region and a universal primer set for the eukaryotic ITS1 region, so as to obtain an amplicon library for each sample. Amplicons were sequenced using MiSeq (Illumina), and the results were analyzed using the DADA2 pipeline.

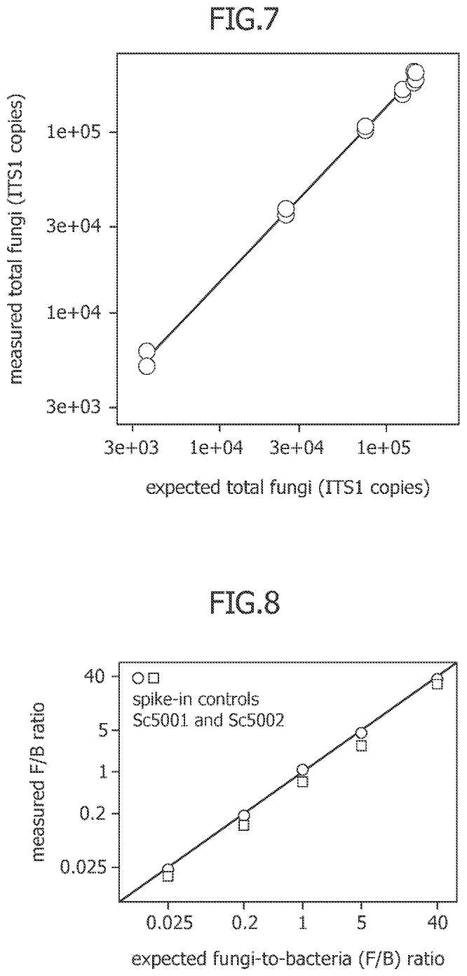

FIGS. 7 and 8 show the results. In FIG. 7, the horizontal axis indicates the estimated copy number of the ITS1 region per unit of artificial sequence, the vertical axis indicates the measured copy number of the ITS1 region. In FIG. 8, the horizontal axis indicates the estimated fungi/bacteria mixing ratio, and the vertical axis indicates the measured fungi/bacteria mixing ratio. In addition, “Sc5001” indicates nucleic acid 3 (SEQ ID NO: 60), and “Sc5002” indicates nucleic acid 4 (SEQ ID NO: 61). These results showed that metagenomic analysis using the nucleic acids 3 to 10 as internal standard nucleic acids can accurately estimate the fungal/bacterial abundance ratio in a sample.

Next, a sample was prepared by adding the nucleic acid 4 (8.3 to 8.3×10³ copies) to DNA (1 ng) extracted from soil, and PCR was performed under the same conditions as in 1 above using a universal primer set for the prokaryotic 16S rRNA V4 region and a universal primer set for the eukaryotic ITS1 region, so as to obtain an amplicon library for each sample. Amplicons were sequenced using MiSeq (Illumina) and the results were analyzed using the DADA2 pipeline.

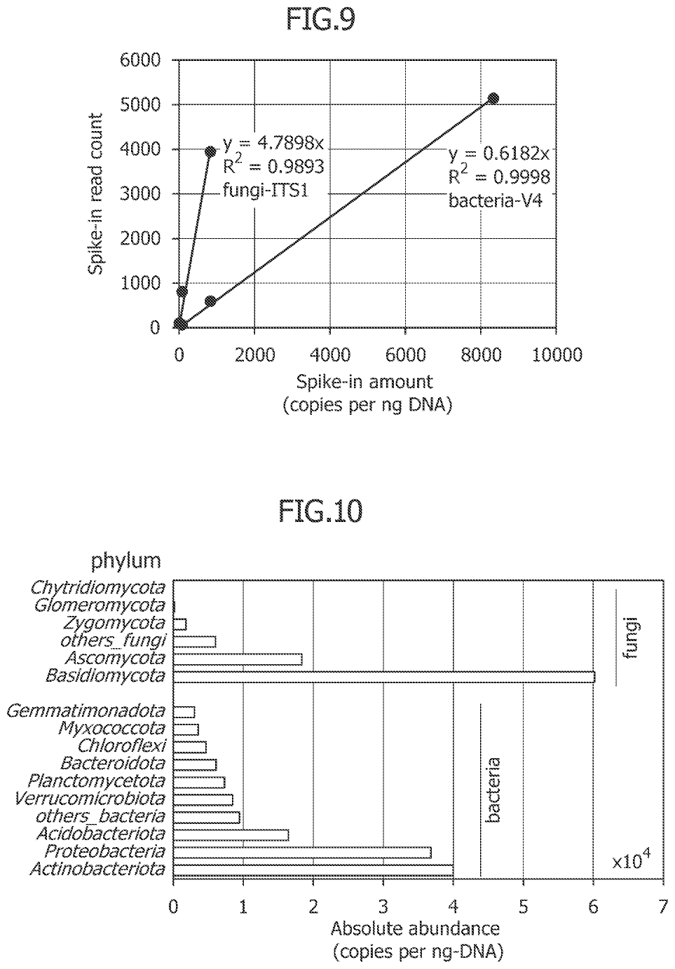

FIG. 9 shows the number of reads derived from the nucleic acid 4 when the total number of reads was made the same for the amount of the nucleic acid 4 added. For both the universal primer set for the prokaryotic 16S rRNA V4 region and the universal primer set for the eukaryotic ITS1 region, there was a high correlation between the amount of nucleic acid 4 added and the read counts. Also, FIG. 10 shows the abundance (absolute number) of microorganisms for each phylogenetic classification (phylum), estimated based on the number of reads derived from the nucleic acid 4. It was demonstrated that it is possible to estimate the absolute abundance of fungi/bacteria in a sample by using the nucleic acid 4 as an internal standard nucleic acid.

1. A nucleic acid comprising at least one partial nucleic acid sequence and/or a complementary sequence thereof, the partial nucleic acid sequence consisting of:
(1) a 5′ flanking sequence comprising a nucleic acid sequence derived from a eukaryotic rRNA-related gene;
(2) an artificial nucleic acid sequence consisting of a non-naturally occurring nucleic acid sequence; and
(3) a 3′ flanking sequence comprising a nucleic acid sequence derived from a eukaryotic rRNA-related gene,
wherein the partial nucleic acid sequence is selected from the group consisting of partial nucleic acid sequences (a) to (d) below:
a partial nucleic acid sequence (a) consisting of:
(a1) a 5′ flanking sequence comprising at least 20 continuous nucleotides in the nucleic acid sequence of SEQ ID NO: 1;
(a2) an artificial nucleic acid sequence consisting of any one of the nucleic acid sequences of SEQ ID NOs: 8 to 19; and
(a3) a 3′ flanking sequence comprising at least 20 continuous nucleotides in the nucleic acid sequence of SEQ ID NO: 2;
a partial nucleic acid sequence (b) consisting of:
(b1) a 5′ flanking sequence comprising at least 20 continuous nucleotides in the nucleic acid sequence of SEQ ID NO: 2;
(b2) an artificial nucleic acid sequence selected from the group consisting of SEQ ID NOs: 20 to 31; and
(b3) a 3′ flanking sequence comprising at least 20 continuous nucleotides in the nucleic acid sequence of SEQ ID NO: 3;
a partial nucleic acid sequence (c) consisting of:
(c1) a 5′ flanking sequence comprising at least 20 continuous nucleotides in the nucleic acid sequence of SEQ ID NO: 3;
(c2) an artificial nucleic acid sequence selected from the group consisting of SEQ ID NO: 32 to 43; and
(c3) a 3′ flanking sequence comprising at least 20 continuous nucleotides in the nucleic acid sequence of SEQ ID NO: 4; and
a partial nucleic acid sequence (d) consisting of:
(d1) a 5′ flanking sequence comprising at least 20 continuous nucleotides in the nucleic acid sequence of SEQ ID NO: 4;
(d2) an artificial nucleic acid sequence selected from the group consisting of SEQ ID NOs: 44 to 55; and
(d3) a 3′ flanking sequence comprising at least 20 continuous nucleotides in the nucleic acid sequence of SEQ ID NO: 5.

2. The nucleic acid according to claim 1, wherein
the partial nucleic acid sequence (a) consists of:
(a1′) a 5′ flanking sequence comprising the nucleic acid sequence of SEQ ID NO: 1;
(a2) an artificial nucleic acid sequence consisting of any one of the nucleic acid sequences of SEQ ID NOs: 8 to 19; and
(a3′) a 3′ flanking sequence comprising the nucleic acid sequence of SEQ ID NO: 2;
the partial nucleic acid sequence (b) consists of:
(b1′) a 5′ flanking sequence comprising the nucleic acid sequence of SEQ ID NO: 2;
(b2) an artificial nucleic acid sequence selected from the group consisting of SEQ ID NOs: 20 to 31; and
(b3′) a 3′ flanking sequence comprising the nucleic acid sequence of SEQ ID NO: 3;
the partial nucleic acid sequence (c) consists of:
(c1′) a 5′ flanking sequence comprising the nucleic acid sequence of SEQ ID NO: 3;
(c2) an artificial nucleic acid sequence selected from the group consisting of SEQ ID NO: 32 to 43; and
(c3′) a 3′ flanking sequence comprising the nucleic acid sequence of SEQ ID NO: 4; and/or
the partial nucleic acid sequence (d) consists of:
(d1′) a 5′ flanking sequence comprising the nucleic acid sequence of SEQ ID NO: 4;
(d2) an artificial nucleic acid sequence selected from the group consisting of SEQ ID NOs: 44 to 55; and
(d3′) a 3′ flanking sequence comprising the nucleic acid sequence of SEQ ID NO: 5.

3. The nucleic acid according to claim 1, further comprising an additional partial nucleic acid sequence (e) and/or a complementary sequence thereof, the additional partial nucleic acid sequence (e) consisting of:
(e4) a 5′ flanking sequence comprising a nucleic acid sequence derived from a prokaryotic rRNA gene;
(e5) an artificial nucleic acid sequence consisting of a non-naturally occurring nucleic acid sequence; and
(e6) a 3′ flanking sequence comprising a nucleic acid sequence derived from a prokaryotic rRNA gene.

4. The nucleic acid according to claim 3, wherein the additional partial nucleic acid sequence (e) consists of:
(e4′) a 5′ flanking sequence comprising the nucleic acid sequence of SEQ ID NO: 6;
(e5′) an artificial nucleic acid sequence of SEQ ID NO: 56 or 57; and
(e6′) a 3′ flanking sequence comprising the nucleic acid sequence of SEQ ID NO: 7.

5. The nucleic acid according to claim 1, consisting of a nucleic acid sequence selected from the group consisting of SEQ ID NOs: 58, 59, and 62 to 69 and/or a complementary sequence thereof.

6. The nucleic acid according to claim 3, consisting of the nucleic acid sequence of SEQ ID NO: 60 or 61 and/or a complementary sequence thereof.

7. An expression vector comprising the nucleic acid according to claim 1.

8. A transformed cell comprising the expression vector according to claim 7.

9. A probe comprising a nucleic acid sequence or a complementary sequence thereof, wherein the nucleic acid sequence is at least 90% identical to a nucleic acid sequence comprising at least 15 continuous nucleotides in an artificial nucleic acid sequence selected from the group consisting of SEQ ID NOs: 8 to 57.
